(19) United States
(12) Patent Application Publication
(10) Pub. No.: US 2012/0277573 A1
(43) Pub. Date: Nov. 1, 2012

(54) ULTRASOUND-ACTIVATED NANOPARTICLES AS IMAGING AGENTS AND DRUG DELIVERY VEHICLES

(76) Inventors: Andy Y. Chang, Pasadena, CA (US); Travis J. Williams, Los Angeles, CA (US); Emine Boz, Los Angeles, CA (US)

(21) Appl. No.: 13/517,995

(22) PCT Filed: Dec. 24, 2010
(86) PCT No.: PCT/US2010/062104

§ 371 Date: Jun. 21, 2012

Related U.S. Application Data
(60) Provisional application No. 61/290,053, filed on Dec. 24, 2009.

(51) Int. Cl. A61K 9/14; A61K 39/395; A61M 37/00; A61B 8/13; A61B 5/055; A61K 49/00; A61B 6/00

(52) U.S. Cl. 600/420; 424/490; 424/9.1; 424/130.1; 600/431; 604/22

(57) ABSTRACT

The invention provides nanoparticles for delivery of imaging agents, drugs, and other molecules, such as genetic material. The nanoparticles have a core structure comprising the imaging agent and/or drug, and a shell structure that allows for water solubility. The shell structure further provides a barrier with limited water permeability that protects the core. The nanoparticles can be induced to release their cargo by treatment with ultrasound. Methods of delivering drugs and imaging agents are also provided, whereby the nanoparticles are delivered to tissues of interest in a substantially inert form, then activated using ultra-sound to release the drugs or imaging agents.

### CROSS-REFERENCE TO RELATED APPLICATIONS

This application relies on and claims the benefit of the filing date of U.S. provisional patent application No. 61/290,053, filed 24 Dec. 2009, the entire disclosure of which is incorporated herein by reference.

### BACKGROUND OF THE INVENTION

1. Field of the Invention

The present invention relates to the fields of nanotechnology and medicine. More specifically, the invention relates to nanoparticles for use in medical diagnostics, evaluation, and treatment of patients.

2. Discussion of Related Art

Numerous agents are known in the art for imaging of tissues and organs of animals. In addition, numerous vehicles for delivery of such agents to the tissues and organs are known in the art. Likewise, numerous bioactive agents and molecular probes are known for therapeutic or prophylactic treatment of animals suffering from, being pre-disposed to, or at risk of developing various diseases and disorders.

For example, imaging agents that are detectable using X-ray technologies (e.g., X-rays, CT/CAT scans) and magnetic resonance imaging (MRI) are well known and widely used in the medical diagnostics field. Broadly speaking, the agents possess a property that can be detected by a particular detection device. When introduced into the body of a patient (used interchangeably herein with “subject” and “animal”), the presence of the agent at a site of interest (e.g., a target tissue) allows an image of the site to be created, thus allowing the medical practitioner to view and assess the site. Use of such agents is possible in numerous diseases and disorders, and for a wide range of tissues and organs in animals.

While it is possible to use such agents directly, it is common to combine the agents with other substances or complex the agents with other substances to improve the half-life of the agent in the patient or to target the agent to a particular organ, tissue, or cell type. Various designs for delivery vehicles for agents have been published and patented, many involving technologies to reduce clearance of the vehicles (and thus agents) by the liver. Many such vehicles are nanoparticles that complex the agent with molecules that sequester or otherwise protect the agent from degradation and clearance from the patient's body. For example, a publication by Parac-Vogt et al. (Parac-Vogt, T. N.; Kimpe, K.; Laurent, S.; Piérart, S.; Vander Elst, L.; Muller, R. N.; Binnemans, K. Gadolinium DTPA-Monoamide Complexes Incorporated into Mixed Micelles as Possible MRI Contrast Agents. Eur. J. Inorg. Chem. 2004, 3538-3543) discloses a hybrid particle featuring a non-covalent core composed of phospholipids and functionalized gadolinium monomers coated with a shell composed of polysorbitol-20 (Tween-80).

While there are numerous agents and delivery vehicles available for diagnostic and therapeutic uses, the present inventors have recognized that there still exists a need in the art for vehicles that can target and deliver imaging agents, bioactive agents, molecular probes, and the like to organs, tissues, and cells of animals.

### SUMMARY OF THE INVENTION

The present invention provides a nanoparticle delivery vehicle that can be used selectively to deliver an imaging agent, a bioactive agent, a molecular probe, or other substance to an area of an animal's body, including a pre-selected organ, tissue, or cell type. The nanoparticle delivery vehicle (used interchangeably herein with reference to the present invention with “nanoparticle”) is particularly well suited for delivery of imaging agents to organs, tissues, and cells of interest for diagnosis and prognosis of diseases and disorders affecting or involving such organs, tissues, and cells. The nanoparticle is also particularly well suited for delivery of bioactive agents, such as cytotoxins, anti-viral agents, and anti-parasitic agents, to target cells to treat or prevent diseases and disorders, including infections and malignancies.

In general, the nanoparticle includes a core structure composed of organic or metallic material (or a combination thereof), a shell structure that adheres to the core structure in a way that it is bound firmly to the core in aqueous solution; and a cargo that the nanoparticle is capable of carrying. The constituent parts of the core structure are bound to each other by covalent or non-covalent chemical interactions. Where the core structure comprises metallic material (e.g., a metal atom, metallic cluster or colloid), preferably some or all of the interactions are covalent bonds. In addition, the constituent parts of the shell structure are bound to each other by covalent or non-covalent chemical interactions. Preferably, some or all of the interactions are covalent bonds.

One noteworthy feature of the nanoparticle design is that the bonds that adhere the core structure to the shell structure can be broken by input of energy from a source external to the subject's body, such as electromagnetic energy (e.g., radio waves, microwaves) or, preferably, mechanical energy (e.g., ultrasound). As such, the core structure and shell structure can be controllably separated when properly treated with the appropriate type and level of energy.

Yet another noteworthy feature of the nanoparticle design is that, when attached to the core structure, the shell structure limits or prevents interaction of the cargo with the external aqueous environment by way of sequestering the cargo within a water-resistant (i.e., semi-permeable) or water-impermeable barrier. For ease of reference, this barrier is referred to herein at times as a “hydrophobic barrier”. Dissociation of all or part of the shell structure from the core structure removes or impairs this hydrophobic barrier and allows the cargo to interact with the aqueous environment.

The present invention also provides methods of using the nanoparticles of the invention. In general, the methods can be any methods in which an imaging agent (used herein interchangeably with “contrasting agent”), a bioactive agent, a molecular probe, or the like is used. For example, the method can be a method of delivering an imaging agent to an organ, tissue, or cell to be imaged. The method thus can include the following steps: a) administering to an animal a nanoparticle according to the invention; and b) allowing adequate time for the nanoparticle to locate to an organ, tissue, or cell of interest. Preferably, the method further comprises: c) subjecting the nanoparticle to energy in an amount sufficient to break the chemical bond between the core structure and the shell structure, causing the core structure and shell structure to dissociate. Where desired, the method can be extended to make it a method of imaging a target organ or tissue by including the additional step of using an imaging device that is compatible with the imaging agent to create an image of the target organ or tissue. Preferably, dissociation of the shell structure from the core structure does not cause or result in dissociation of the imaging agent from the core structure.

Alternatively, the method can be a method of delivering a bioactive agent, such as a drug, to an animal organ, tissue, or cell of interest. The method thus can include the following steps: a) administering to an animal a nanoparticle according to the present invention; and b) allowing adequate time for the nanoparticle to locate to an organ, tissue, or cell of interest. Preferably, the method further comprises: c) subjecting the nanoparticle to energy in an amount sufficient to break the chemical bond between the core structure and the shell structure, causing the core structure and shell structure to dissociate. In exemplary embodiments, dissociation of the core structure and the shell structure causes the bioactive agent to dissociate from both of those structures as well. The proximity of the nanoparticle to the organ, tissue, or cell of interest results in a relatively high concentration of the bioactive agent close to the organ, tissue, or cell, and thus results in delivery of the bioactive agent to the organ, tissue, or cell of interest. Because delivery of a bioactive agent can cause a desired clinical effect, the method can be a method of treating a subject suffering from, suspected of suffering from, or at risk of developing a disease or disorder.

Yet again, the method can be a method of delivering a molecular probe, such as a cell-type specific labeling agent, to an animal organ, tissue, or cell. The method thus can include the following steps: a) administering to an animal a nanoparticle according to the present invention; and b) allowing adequate time for the nanoparticle to locate to an organ, tissue, or cell of interest. Preferably, the method further comprises: c) subjecting the nanoparticle to energy in an amount sufficient to break the chemical bond between the core structure and the shell structure, causing the core structure and shell structure to dissociate. In exemplary embodiments, dissociation of the core structure and the shell structure causes the molecular probe to dissociate from both of those structures as well. The proximity of the nanoparticle to the organ, tissue, or cell of interest results in delivery of the molecular probe to the organ, tissue, or cell of interest.

The present invention further provides methods of making the nanoparticles of the invention. In general, the methods include: synthesizing the substances that comprise the nanoparticle, and combining the substances in an order that results in a functional nanostructure. It is to be understood that the order of synthesis is not critical, and the practitioner may elect to perform the recited syntheses in any desired order. It is also to be understood that it is not necessary to synthesize all of the substances prior to initiation of the combining step, and that certain substances may be combined separately, then the combinations combined with other substances or combinations. It is yet further to be understood that the term “synthesizing” includes the act of obtaining pre-synthesized substances, for example from a commercial vendor. In exemplary embodiments, the method of making includes: synthesizing a core structure, combining the core structure with a cargo, and combining the core structure/cargo with constituent components of the shell structure. As such, in embodiments the shell structure is not synthesized as a complete unit prior to combining with the core structure, the cargo, or both. Rather, the shell may be synthesized as a result of binding of its constituent components to the core structure.

Ancillary to the methods of making the nanoparticles of the invention, a method for the chemical synthesis of highly fluorinated amines and diamines is provided. In general, the method includes: a) converting tetraethyleneglycol monomethyl ether to the tosylate; b) converting the tosylate to a mono-alkylated product by reacting the tosylate with fluorinated diol in the presence of sodium hydride; c) converting the alcohol to an amine functionality by formation of the triflate and displacement with potassium phthalimide, to form a carbon-nitrogen bond; and d) reducing the product with hydrazine to form a highly fluorinated amine. The highly fluorinated amines and diamines find use within the context of the present invention as the hydrophobic barrier of the shell structure. Details of the synthetic process are provided in the Examples below.

The present invention has wide applicability and utility in the fields of medical diagnosis and treatment. Non-limiting examples include: the use in patients undergoing a Voiding Cystourethrogram (VCUG); imaging the selective delivery of ultrasound to living tissue or other aqueous media; and selective imaging and drug delivery to tumors. In general, the invention is applicable to all situations where delivery of contrast/imaging agents, therapeutic agents, or molecular probes to any tissue is desired, potentially with release of the agent(s) using externally-supplied energy, such as ultrasound, to achieve site-specific detection and, in embodiments, delivery, of the agent(s).

Further, the invention includes, but is not limited to, the following additional uses of the nanoparticles of the invention: providing MRI contrast in vivo by treatment of tissue containing the nanoparticles of the invention with ultrasound; diagnosis and surveillance of vesicoureteral reflux disease (VUR); catheter-free cystography; the delivery of a drug or molecular probe to a locus selected by application of ultrasound radiation. It yet further includes, but is not limited to, the development of polyamide (nylon) materials featuring a fluorous diamine region. Of course, the invention contemplates any and all combinations of the applications discussed herein.

### BRIEF DESCRIPTION OF THE DRAWINGS

The accompanying drawings, which are incorporated in and constitute a part of this specification, illustrate several embodiments of the invention, and together with the written description, serve to explain certain principles of the invention.

FIG. 1 shows a diagrammatic representation of a nanoparticle of the invention, showing a dual imaging and drug delivery particle.

FIG. 2 shows a schematic of preparation and use of a nanoparticle of the invention as a contrasting agent for VCUG.

FIG. 3 depicts an experimental design for preparation of a contrasting agent nanoparticle according to the invention.

FIG. 4 depicts exemplary materials from which nanoparticles according to the invention can be made.

FIG. 5 depicts proposed reasons for low enhancement of contrast using a particular nanoparticle.

FIG. 6, Panels A-C, depict exemplary materials from which an improved nanoparticle according to the invention can be made, and its properties.

FIG. 7, Panels A-C, show bar graphs depicting fluctuations in particle size distributions upon addition and removal of an arginine shell. A: (Particle) r1=22.5 nm (3.4% Intensity), r2=137.1 (96.6% Intensity). B. (Particle with arginine) r=130.7 nm. C. (Particle with Arginine and urea) r1=32.1 nm (11.7% Intensity), r2=129.7 nm (88.3% Intensity). For all, r=diameter.

FIG. 8, Panels A and B, depict synthesis schemes for a shell molecule containing a fluorous hydrophobic “raincoat” region.

FIG. 9 depicts a synthetic scheme for a particle built on a gold-based scaffold.

FIG. 10 depicts a design for a removable shell. Panel A shows shell molecule design. Panel B shows a chemical strategy for shell removal.

FIG. 11 depicts a synthesis scheme for a gold-based nanoparticle.

FIG. 12 depicts a scheme for preparing photo-crosslinked core structures.

FIG. 13 depicts a synthesis scheme for bisphosphonate-functionalized thiols.

### DETAILED DESCRIPTION OF THE INVENTION

Reference will now be made to various exemplary embodiments of the invention, examples of which are illustrated in the accompanying drawings. It is to be understood that the following description is provided to assist the reader in understanding certain details and features of embodiments of the invention and should not be considered as a limitation on the scope or content of the invention. For example, while the following description focuses on the urinary system, the concepts, agents, and methods are equally applicable to other systems, organs, and tissues.

Before embodiments of the present invention are described in detail, it is to be understood that the terminology used herein is for the purpose of describing particular embodiments only, and is not intended to be limiting. Further, where a range of values is provided, it is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention. Further, in accordance with generally accepted terminology, the term “nanoparticle” means particles having a size between one and one thousand nanometers (nm).

Unless defined otherwise, all technical and scientific terms used herein have the same meaning as commonly understood by one of ordinary skill in the art to which the term belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. The present disclosure is controlling to the extent it conflicts with any incorporated publication.

As used herein and in the appended claims, the singular forms “a”, “an”, and “the” include plural referents unless the context clearly dictates otherwise. Thus, for example, reference to “a nanoparticle” includes a plurality of such particles and reference to “a cell” includes reference to one or more cells and equivalents thereof known to those skilled in the art, and so forth. Furthermore, the use of terms that can be described using equivalent terms include the use of those equivalent terms. Thus, for example, the use of the term “subject” is to be understood to include the terms “animal”, “human”, and other terms used in the art to indicate one who is subject to a medical treatment. As another example, the use of the term “neoplastic” is to be understood to include the terms “tumor”, “cancer”, “aberrant growth”, and other terms used in the art to indicate cells that are replicating, proliferating, or remaining alive in an abnormal way.

The present invention relates to nanoparticles that are “activatable” by absorption of energy, such as by ultrasound. Specifically, nanoparticles comprising one or more bioactive agents (e.g., drugs), agents for imaging tissues and organs (e.g., contrasting agents for MRI), or other agents having utility in medical treatments and clinical diagnostics are provided, where the nanoparticles have a structure in which the agents are encapsulated or coated with one or more substances that render the particles inert or inactive for their intended purpose (e.g., biological activity, imaging agent). The particles are treated with energy, e.g., ultrasound, to expose the agents to the external environment when and where desired, thereby providing the desired activity at the desired site.

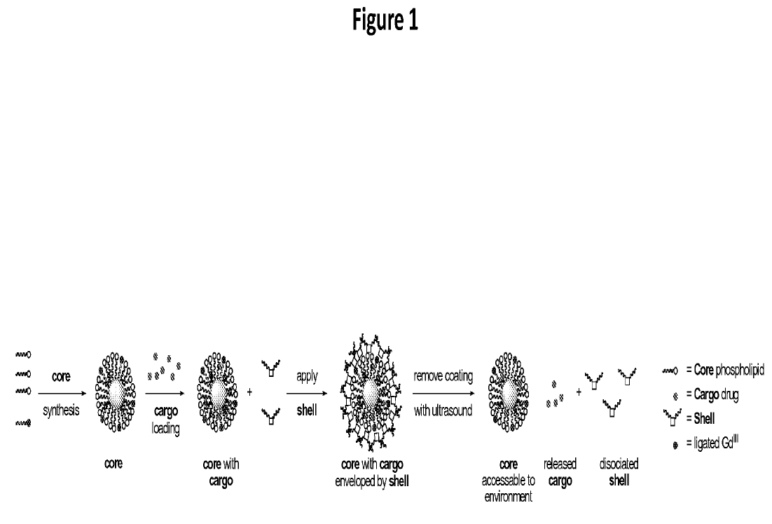

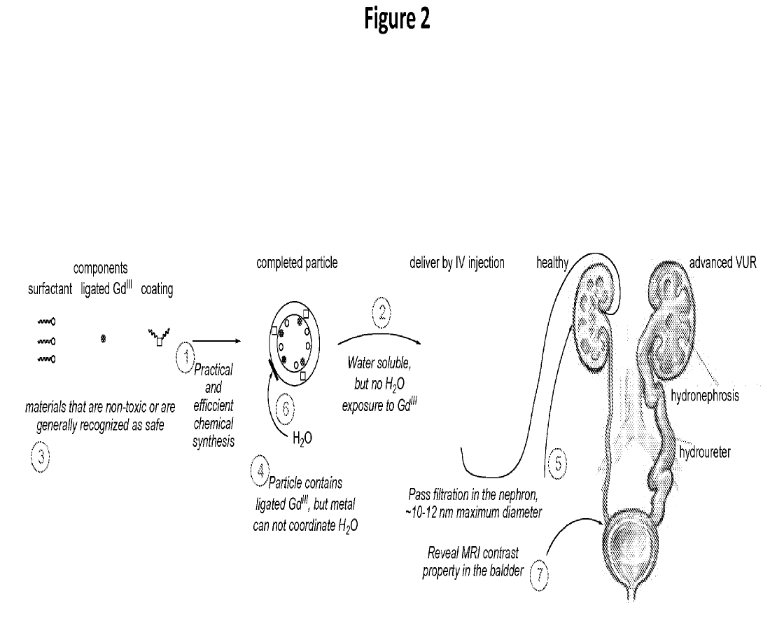

The nanoparticles can be designed to include any substance of interest (referred to herein as “cargo”) that is desired to be delivered to a tissue without the substance being exposed to the environment of the body into which it is delivered. That is, nanoparticles of the invention can include any substance that is desired to be protected until it is delivered to the site of interest. In exemplary embodiments, the nanoparticles include imaging agents for diagnostic or other clinical purposes. In other exemplary embodiments, the nanoparticles include bioactive agents for therapeutic and/or prophylactic purposes. In some exemplary embodiments, the nanoparticles include both imaging agents and bioactive agents. The general process for preparation and use of a nanoparticle according to the invention, which includes both an imaging agent and a bioactive agent, is depicted in FIG. 1.

The invention provides a nanoparticle comprising a core structure having an organic material, a metal-containing material, or both, wherein the core structure comprises an inner core region for forming the core structure, and an outer core region for bonding the core structure to a shell structure. The nanoparticle also comprises a shell structure bound to the core structure, wherein the shell structure comprises, in sequential arrangement: a binding region for binding to the core structure; a hydrophobic region for protection of the binding region and core structure from hydrophilic substances, and a hydrophilic region for rendering the nanoparticle soluble in aqueous environments. Preferably, the hydrophobic region is also lipophobic. The nanoparticle further comprises a cargo, which can be any substance that the practitioner desires to deliver to a target within a patient. While not so limited, in exemplary embodiments, the cargo is a detectable agent (e.g., an MRI contrasting agent), a bioactive agent (e.g., a drug), a molecular probe, or a combination of two or all three of these classes of molecules.

As stated above, the core structure comprises an inner core region. The inner core region defines the portion of the nanoparticle where the outer core region molecules are physically linked to each other, either directly or indirectly, to form the core structure. In embodiments, the inner core region comprises a metal that can form covalent bonds with the organic compounds that comprise the outer core region. In such embodiments, the outer core region molecules are physically linked to each other by way of their bonding to the metal. In exemplary embodiments, the metal is gold. In embodiments, the metal is not iron.

In some embodiments, the inner core region does not comprise a metal. Rather, in some embodiments, the inner core region comprises another substance (e.g., element, organic compound, inorganic compound) that serves the function of binding and physically linking the outer core region molecules. The substance is not limited in structure and can be selected by the practitioner based on any number of parameters. Likewise, the inner core region can be occupied only by outer core region molecules. In such a configuration, the outer core region molecules interact directly with each other at the inner core region. For example, the outer core region molecules can interact with each other by way of non-covalent bonding, such as through hydrophobic interactions. Likewise, they can interact with each other or with another substance through chemical cross-linking as a result of exposure to energy, such as electromagnetic radiation (e.g., ultraviolet light) or mechanical energy (e.g., ultrasound). Alternatively, they can contain a reactive group at one terminus that allows for interaction and bonding to other outer core region molecules or another substance. The types of interactions are not critical as long as the interactions are sufficiently strong to maintain a linkage between the outer core region molecules during synthesis and use of the nanoparticles.

In general, the inner core is spherical or substantially spherical. The size of the inner core may be varied to suit particular applications of the technology. For example, for delivery of imaging agents, the inner core can be on the order of 1 nm to 5 nm in diameter, whereas for delivery of bioactive agents or molecular probes, the inner core can be on the order of 5 nm to 25 nm in diameter. It is to be understood that other sizes outside of these exemplary ranges may be used as well.

The inner core region is surrounded by the outer core region. The outer core region comprises molecules that link the inner core region to the shell. The type of molecule used for the outer core region molecules is not particularly limited, with the exception that it should be able to form a sufficiently strong linkage to a metal, to other outer core molecules, or to another substance at the inner core region to maintain the integrity of the core structure during fabrication and use. In embodiments, the outer core molecule forms a covalent bond on one end with a metal comprising the inner core region, and forms a non-covalent bond (e.g., a hydrogen bond) on the other end with a molecule comprising the shell structure. In exemplary embodiments, the outer core region comprises organic molecules, such as phosphonic acid surfactants, which are capable of covalently bonding to a metal, such as gold, at the inner core region, and also capable of bonding to the molecules that comprise the shell (discussed in more detail below). In exemplary embodiments, the organic molecules of the outer core region and the metal of the inner core region bond as a result of a sulfhydryl group at one terminus of the organic molecules.

The size or length of the outer core region will vary depending on the intended use of the nanoparticle. For example, for delivery of imaging agents to the kidney, the outer core region will be on the order of 5 nm to 9 nm, allowing for an overall core structure of 10 nm or less in diameter. Alternatively, for delivery of certain imaging agents to other tissues or organs, the outer core region can be on the order of 5 nm to 200 nm. Yet again, for delivery of imaging agents, bioactive agents, and molecular probes by way of release of these substances from the core structure upon dissociation of the shell structure, the outer core region can be on the order of 5 nm to 200 nm or more, with the understanding that more imaging agent, bioactive agent, or molecular probe can be loaded into the core structure as the length of the outer core region is increased. The design of the nanoparticle should take into account the total size of the particle and its intended use. Thus, for example, for use in vesicoureteral reflux, the core and shell are designed in conjunction with each other such that the total nanoparticle diameter is 10 nm or less. Likewise, for chemotherapeutic applications, the core and shell are designed together to have a total diameter of, for example, 200 nm.

The outer core region also comprises a cargo. A cargo according to the present invention is any substance that the practitioner desires to be delivered to a target site by the nanoparticle delivery vehicle of the invention. While not so limited in structure or function, three general classes of exemplary molecules are discussed herein: imaging or contrasting agents for clinical/medical diagnostics; bioactive agents for treatment of patients; and molecular probes. Where provided for diagnostics, the cargo is preferably a substance that can be detected using one or more commercially available systems. It thus may be any of the commercially available imaging agents known in the art, such as those agents having paramagnetic properties. In exemplary embodiments, the imaging agent comprises a thiol-terminated paramagnetic substance, such as a gadolinium (e.g., Gd^{III}) or iron containing substance.

One advantage provided by the nanoparticle delivery vehicles of the present invention is the use of imaging agents that can be used with systems that do not rely on X-rays for detection. While X-ray detection is common and widely practiced, the ability to avoid using X-rays, and to avoid the collateral damage they can cause, is a distinct advantage that is provided by this invention.

Cargo according to the invention can also or alternatively be bioactive. As used herein, a bioactive agent is any substance that has a biological or biochemical effect on a subject to whom it is administered. The number and identity of bioactive agents encompassed by the present invention is vast, and is not particularly limited by structure or function. Non-limiting examples include small molecule drugs (e.g., chemotherapeutic agents, anti-inflammatory agents), biologicals (e.g., therapeutic peptides, polypeptides, proteins), antibodies, antigens, cytotoxins, hormones, nucleic acids (e.g., anti-sense DNA molecules, siRNA, microRNA, protein-encoding dsDNA), and anti-viral agents. Those of skill in the medical art can envision numerous other bioactive agents, the listing of all of which would not be practical within this document. All such agents are contemplated by the present invention.

Depending on the intended use of the cargo, it can be chemically bonded to the inner core or the outer core molecules, or can be freely associated with the outer core molecules. For example, where the cargo is an imaging agent that does not require release from the nanoparticle structure for activity, the imaging agent can be chemically (e.g., covalently) bound to the inner core region (e.g., gold particle). In contrast, where the cargo is a bioactive agent that functions inside a target cell, the bioactive agent can be freely associated with the outer core molecules such that, upon removal of the shell, the bioactive agent can diffuse out from the core and enter the target cell.

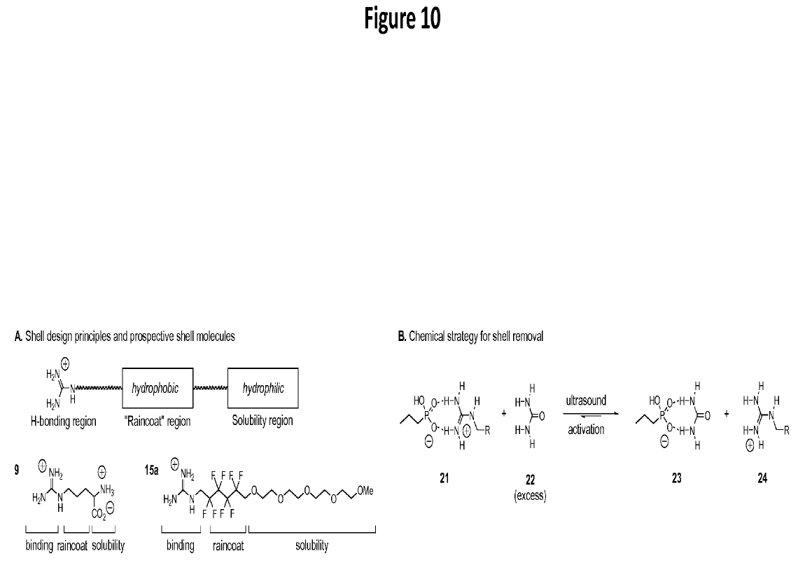

In the nanoparticle delivery vehicle, the core structure is surrounded by a shell structure. The shell structure comprises molecules having three distinct regions arranged in the following sequential order: a bonding region for bonding to the outer core region molecules, a hydrophobic “raincoat” region, and a hydrophilic region. The unique design of the shell molecules provides, in a single molecule, the ability to bond to the core structure a molecule that possess a water-resistant or water impermeable layer surrounded by a water soluble layer. When bound to the core region, the nanoparticle delivery vehicle is water soluble, yet at the same time protects its cargo from interaction with water. Such a design allows for delivery of water labile cargoes to their intended sites of action without significant degradation of the cargoes. It also allows for delivery of cargo without loss of cargo during delivery by diffusion from the delivery vehicle. Likewise, the design allows for activation of water-activated cargoes only upon removal of the shell. An exemplary shell molecule is depicted in FIG. 10, which is discussed in detail below.

The bonding region of the shell molecule provides a structure that is suitable for and capable of bonding to one end of the inner core region molecules. It is thus not particularly limited in structure, although it must be designed in conjunction with the outer core region molecules to allow bonding of the two. Preferably, the bonding is through one or more (e.g., two) hydrogen bonds per linkage. Numerous chemical groups that can bond to each other are known in the chemistry art, and any such groups can be used. The chemical groups also must be designed such that the bonds linking the outer core region molecules to the shell molecules can be broken through the use of energy supplied from a source located outside of the body in which the nanoparticles are introduced. For example, where the practitioner desires to use ultrasound to “activate” the nanoparticles, the bond holding the outer core molecules to the shell molecules must be one that can be broken using ultrasound energy. Of course, when used in vivo, the bonds must break before significant or irreparable damage is done to the body of the patient. In exemplary embodiments, the core and shell are bound through the interaction of a guanidinium cation and phosphate or carboxylate anion or an analogous interaction.

The shell molecules are attached to the phosphonic acid periphery by a guanidinium-phosphonate double hydrogen bonding system similar to the one viruses such as HIV-tat utilize to endocytose or otherwise enter cells (Frankel, A. D.; Pabo, C. O. Cellular uptake of the tat protein from human immunodeficiency virus. Cell 1988, 55, 1189-1193; Wender, P. A.; Mitchell, D. J.; Pattabiraman, K.; Pelkey, E. T.; Steinman, L.; Rothbard, J. B. The Design, Synthesis, and Evaluation of Molecules That Enable or Enhance Cellular Uptake: Peptoid Molecular Transporters. Proc. Natl. Acad. Sci. U.S.A. 2000, 97, 13003-13008; Rothbard, J. B.; Jessop, T. C.; Lewis, R. S.; Murray, B. A.; Wender, P. A. Role of Membrane Potential and Hydrogen Bonding in the Mechanism of Translocation of Guanidinium-Rich Peptides into Cells J. Am. Chem. Soc. 2004, 126, 9506-9507). In this case, hydrogen bonding between the phosphonic acid periphery and a guanidine functionality at the terminus of the shell molecule ensures tight binding as has been observed in other micelle applications (Paleos, C. M.; Kardassi, D.; Tsiourvas, D. Transformation of Dihexadecyl Phosphate Vesicles to Micelles Using Guanidinium-Type Counterions. Langmuir 1999, 15, 282-284). In this application the association constant is large, Ka˜102-104 M-1 (Fokkens, M.; Schrader, T.; Klrner, F.-G. A Molecular Tweezer for Lysine and Arginine. J. Am. Chem. Soc. 2005, 127, 14415-14421; Williams, G.; Hardly, M. L. The Ionisation of Guanidine and of Amino-Acids in Sulphuric Acid. J. Chem. Soc. 1953, 2560-2563). In this scheme, hydrogen bonding and a Columbic attraction between the phosphonic acid periphery and a guanidinium functionality on the shell molecule ensures tight binding. This specific interaction is cleaved in the body with clinical ultrasound. For kidney applications, the dissociation of shell is advantageously driven to completion by the high concentration of urea (1.0 to 1.5 mM) in the bladder. Specifically, urea in the bladder acts to cap the phosphonic acid groups on the particle and help ensure that the shell molecules do not re-associate.

The bonding region is adjacent the hydrophobic region of the shell molecule. The hydrophobic region can comprise any atom, chemical moiety, chemical group, etc. that has the characteristic of hydrophobicity. In embodiments, it has the characteristic of lipophilicity. In other embodiments, it has the characteristic of lipophobicity. It thus may be a series of hydrophobic amino acids, for example three to twenty residues in length. Alternatively, it may comprise hydrophobic groups commonly found in thermoplastics or thermosetting resins. Yet further, it may comprise an alkane region. Numerous atoms, moieties, groups, etc. are known in the chemical arts, and any one or combination of two or more may be used in accordance with the invention. The non-limiting exemplary embodiment depicted in the figures shows the use of fluorine atoms as the hydrophobic region of the shell molecule. As such, in embodiments the hydrophobic region of the shell structure comprises the following structure: (CF₂)ₙ, wherein n=2-10, such as 3, 4, or 6.

The shell structure comprises numerous shell structure molecules bound via their bonding region to the outer core molecules of the core structure. While not so limited, it is highly preferred that the shell structure comprise numerous molecules all of the same structure. Regardless of whether multiple identical molecules are used or whether molecules having different structures are used, it is important that the shell structure molecules, once bound to the outer core molecules, form an uninterrupted, or substantially uninterrupted, hydrophobic barrier that surrounds or encases the bonding region, the core structure, and the cargo. As such, where shell structure molecules having more than one chemical structure are used, it is important that the distance between the bond between the shell structure molecules and the hydrophobic region be engineered to be the same so that a hydrophobic barrier is created.

A hydrophilic region is located adjacent the hydrophobic region of the shell structure molecule. The hydrophilic region is provided to make the nanoparticle delivery vehicle of the invention soluble in aqueous environments. A water-soluble nanoparticle has obvious advantages over a water-insoluble nanoparticle, particularly when the particle is designed for use in an animal (used herein to include humans). As with the other regions of the shell molecule and the outer core molecule, the hydrophilic region of the shell molecule is not particularly limited in size, length, or chemical make-up. As long as the region confers upon the resulting nanoparticle delivery vehicle the property of hydrophilicity or solubility in aqueous environments (e.g., blood, insterstitial space), it satisfies the criteria. As with hydrophobic atoms, chemical groups, moieties, etc., those of skill in the chemical arts are well aware of the numerous hydrophilic substances known in the art. As with the hydrophobic substances, the practitioner is free to select an appropriate hydrophilic substance to include as part of the shell molecule. In general, the hydrophilic region can comprise one or more structure that is capable of hydrogen bonding with water, causing it to be hydrophilic. Non-limiting examples include: glycerol or glycerol-based molecules, polyethylene glycol (PEG) or PEG-based molecules, or folic acid derivatives.

In embodiments, the shell further comprises one or more substances that target the nanoparticle delivery vehicle to a particular organ, tissue, or cell. Such targeting substances are well known in the art, as are the advantages to being able to specifically deliver a cargo to a chosen cell or cell type. Exemplary targeting substances for use in the present invention include, without limitation, substances such as: peptides, antibodies, ligands for cell-surface receptors, and antigens. The use of targeting substances to specifically target bioactive substances to pre-selected cells or cell types is well known in the medical art, and any suitable targeting substance can be used as part of the nanoparticle delivery vehicle of the present invention with regard not only to delivery of bioactive agents, but to other cargoes as well. Binding of such targets to the shell structure molecules can be accomplished using standard chemical or biochemical reactions without undue or excessive experimentation.

The nanoparticle delivery vehicle of the invention can be spherical or substantially spherical, and can have the following arrangement of elements from the interior to the exterior: core structure inner core region; core structure outer core region; shell structure binding region; shell structure hydrophobic region; shell structure hydrophilic region; and optionally, a specific targeting substance for targeting the nanoparticle delivery vehicle to a particular organ, tissue, cell, or cell type. In such a configuration, the cargo is located interior of the shell structure hydrophobic region, preferably completely or substantially within the outer core region.

The nanoparticles of the invention are typically spherical, having a diameter on the order of 500 nm or less, such as 200 nm, 150 nm, 100 nm, 75 nm, 50 nm, 25 nm, 20 nm, 15 nm, 10 nm, 5 nm, or less. Of course, those of skill in the art will immediately recognize that the particles can be any particular value within this disclosed range, without the need for this document to specifically list all 500 values individually. In embodiments, the particle is preferably less than 15 nm in diameter, more preferably less than 10 nm in diameter. One advantage of the present invention is the creation and use of nanoparticle delivery vehicles on the order of 10 nm or less, which are highly advantageous for use in imaging the kidneys, as they can pass filtration in the kidneys. Those of skill in the art will immediately recognize that the nanoparticles according to the invention will not all have the exact same size, but rather will have a size distribution that will cluster around a particular diameter. It is to be understood that reference herein to a particular size or diameter is intended to encompass this noted size distribution.

The nanoparticle delivery vehicle of the present invention has numerous uses throughout biological systems. Non-limiting exemplary uses disclosed herein focus on the use for delivering imaging agents, bioactive agents, and molecular probes. As such, the invention provides nanoparticles for use in delivering imaging agents to a site to be imaged. It likewise provides nanoparticles for use in imaging an organ, tissue, cell, or cell type of interest. Similarly, it provides nanoparticles for use in delivering bioactive agents to an organ, tissue, cell, or cell type of interest. Yet further, it provides nanoparticles for use in treating an organ, tissue, cell, or cell type of interest. The use in treating can be a use for treating a subject suffering from, susceptible to, or at risk of developing a disease or disorder involving an organ, tissue, cell, or cell type. Additionally, the nanoparticles may be used for delivering molecular probes to an organ, tissue, cell, or cell type of interest.

Use of the nanoparticle delivery vehicles of the invention can be explained in terms of methods of using the nanoparticles of the invention. While the present detailed description of the invention focuses on the use of the particles in vivo, it is to be understood that the particles can also be used ex vivo for therapeutic or prophylactic purposes, and can be used in vitro for research purposes.

In embodiments, the method of using the nanoparticles of the invention is a method of delivering an imaging agent to an organ, tissue, cell, or cell type to be imaged. Typically, the clinician or medical practitioner practicing this aspect of the invention will be interested in using the nanoparticles for diagnosis or prognosis of a disease or disorder. Often, the disease or disorder results in or is a result of morphological changes in an organ, tissue, or cell, which can be imaged using known techniques. For example, the disease might be a tumor, and the medical practitioner is interested in determining the size and shape of the tumor to determine the best course of treatment. Alternatively, the disease or disorder might be due to limited function of an organ, and the medical practitioner is interested in determining the size and shape of the organ, or the blood flow in, around, and through the organ. There are, of course, numerous other applications of imaging technology to the medical profession. The present invention relates to all such applications.

The general steps for delivering an imaging agent are discussed above and again briefly below. In certain embodiments, the nanoparticle delivery vehicle of the invention comprises a targeting substance on its outer surface to specifically target the nanoparticle delivery vehicle to a pre-selected organ, tissue, cell, or cell type. Typically, the method of delivering the imaging agent is practiced so as to image the target organ, tissue, cell, or cell type. As such, the method may include using an imaging device that is compatible with the imaging agent to create an image of the target organ, tissue, cell, or cell type. Many such devices are known in the art, and any suitable one may be used. The practitioner is fully capable of selecting compatible imaging/contrasting agents and imaging devices/systems without a lengthy discussion herein. Preferably, the imaging device is an MRI device and the shell structure of the nanoparticle delivery vehicle is partially or completely separated from the core structure and imaging agent as a result of delivery of ultrasound energy to the area of the patient's body where the organ, tissue, cell, or cell type of interest is located. Additionally, in embodiments where the inner core region of the nanoparticle is gold, the practitioner can image the target organ, tissue, cell, or cell type using a computed tomography (or CT) scanner device, without application of ultrasound energy.

In embodiments, the method of using the nanoparticles of the invention is a method of delivering a bioactive agent to an organ, tissue, cell, or cell type of interest. Typically, the medical practitioner practicing this aspect of the invention will be interested in using the nanoparticles for therapeutic or prophylactic treatment of a patient in need, or suspected of being in need, of such treatment. In contrast to delivering an imaging agent, which results in an image being made of the target, the method of delivering a bioactive agent results in a biological effect on the organ, tissue, cell, or cell type. As with other embodiments of the method of the invention, preferably the method of delivering a bioactive agent uses a nanoparticle delivery vehicle that comprises a specific targeting substance on its surface to target the nanoparticles to a pre-selected organ, tissue, cell, or cell type. Further, it is preferable that, once targeted to a specific organ, tissue, cell, or cell type, the nanoparticles be “activated” through removal of some or all of the shell structure molecules from the outer core molecules, allowing the bioactive agent to be released. It is to be recognized that complete removal of the shell structure molecules is not a required step in the method. Rather, once the nanoparticles are targeted to a chosen target, they may be partially “activated” then allowed to remain at the targeted position (e.g., bound to a target cell). Partial activation allows for slow, long-term delivery of the bioactive agent to the cell.

In yet other embodiments, the method of using the nanoparticles of the invention is a method of delivering a molecular probe, such as a cell-type specific labeling agent, to an organ, tissue, cell, or cell type of interest. The method steps are those used in the method of delivering a bioactive agent, and the same considerations apply with regard to release of the probe from the nanoparticle delivery vehicle.

In an exemplary embodiment, the use of the nanoparticle delivery vehicle of the invention is a method of co-delivery of an imaging agent and a bioactive agent. The method steps are those discussed above with regard to delivery of imaging agents and bioactive agents: a) administering to a subject in need or suspected of being in need a nanoparticle delivery vehicle according to the present invention; and b) allowing adequate time for the nanoparticle delivery vehicle to locate to an organ, tissue, or cell of interest. Preferably, the method further comprises: c) subjecting the nanoparticle to energy in an amount sufficient to break the chemical bond between the core structure and the shell structure, causing the core structure and shell structure to dissociate. In this exemplary embodiment, dissociation of the core structure and the shell structure causes or allows the bioactive agent to dissociate from both of those structures. The proximity of the nanoparticle to the organ, tissue, cell, or cell type of interest results in delivery of the bioactive agent to the organ, tissue, cell, or cell type of interest. Further, dissociation of the shell structure and the core structure exposes the imaging agent to the aqueous environment of the body, allowing for imaging of the organ, tissue, cell, or cell type being treated with the bioactive agent. The delivery of a single nanoparticle delivery vehicle having both an imaging agent and a bioactive agent provides a substantial advantage to the medical practitioner, as he is now able to achieve two goals with a single procedure. Doing so results in fewer procedures performed, real-time collection of data and delivery of bioactive agent, and significant cost savings for the patient. Furthermore, combined delivery of imaging agent and bioactive agent allows the medical practitioner to follow the progression of a disease under treatment and the response of a patient to a treatment regimen. For example, using the combined delivery vehicle, an oncologist can not only deliver an anti-cancer agent to a solid tumor, but can, at the same time, determine the effectiveness of the delivery of the treatment at the tumor site by imaging the tumor's size, shape, or other physical characteristics.

It is important to recognize that delivery of energy to the nanoparticle delivery vehicle (regardless of the cargo that it contains) causes the shell structure to dissociate from the core structure. However, dissociation might not be complete. For example, exposure of nanoparticle delivery vehicles to ultrasound energy can, in some embodiments, result in dissociation of all of the shell structure molecules from all of the outer core molecules. However, it can also result in dissociation of fewer than all of the molecules dissociating, resulting in a partial or incomplete dissociation. In such a situation, successful use of the nanoparticles can still be achieved, as the hydrophobic barrier will still have been breached, at least to some extent, allowing water to enter the core structure and, where the cargo is not covalently bound to the inner core, diffusion of some or all of the cargo from the core structure.

The design of the nanoparticle delivery vehicle of the present invention provides a particle that has an outer layer that is hydrophilic (thus making the particle water soluble) and a core that is protected from water by a hydrophobic barrier. Unlike other nanoparticle delivery vehicles known in the art, the nanoparticle delivery vehicle of the present invention can have a core that is completely protected from water by the hydrophobic barrier (i.e., the hydrophobic barrier can be water impermeable). However, it is to be understood that while it is preferred that the hydrophobic barrier be water impermeable, in some embodiments, the hydrophobic barrier is not a complete barrier to water, but is rather a semi-permeable barrier. This characteristic can result from designing the shell structure molecule to include holes or breaches in the hydrophobic barrier, designing the shell structure molecule to allow for chemical or enzymatic degradation of the shell structure molecule, core molecules, or both, or as a result of normal movement of the molecules about each other within the supercomplex that is the nanoparticle delivery vehicle. Such “leakiness” can result in pre-activation of a certain portion of the cargo or can result in loss of a certain portion of cargo prior to “activation” of the nanoparticles with energy. Such “leakiness” can be used to the practitioner's advantage by providing a method in which the nanoparticle delivery vehicle is delivered to a target, and simply allowed to remain in contact with the target while a bioactive agent (e.g., a cytotoxic agent) slowly diffuses out of the nanoparticle, thus delivering a low dose of agent to the target over an extended period of time.

As used herein, administering is any action that results in the nanoparticles of the invention being present within the body (including the alimentary canal). It thus can be any action commonly known in the art for delivery of bioactive agents and/or imaging agents to patients, including, but not limited to, oral ingestion, injection (of any type), infusion, via mucosal membranes, inhalation, and transdermal. When used to study the kidneys, the present invention allows administration via a route other than catheterization. The amounts to be delivered will vary depending on the route chosen, and can easily be determined by the practitioner using standard medical protocols without undue or excessive experimentation.

The amount of time required for delivery of the imaging agent to the target site will also vary depending on the route of administration chosen. For example, for intravenous administration of a nanoparticle delivery vehicle for imaging of the kidneys, imaging can commence approximately 45 minutes after administration. Of course, the time required for each administration route can be determined using standard protocols without undue or excessive experimentation.

Further, the amount of energy to be delivered to release the shell from the core will vary depending on the type of energy used and the particular atoms involved in the bond. As with the other parameters, the amount of energy to be delivered can be determined for each bonding scheme without undue or excessive experimentation. For example, the amount of energy can be on the order of 5 watts or less. However, for deep tissue or dense tissue, the amount can be higher.

It is believed that this is the first disclosure of a water soluble nanoparticle that can sequester a cargo from an aqueous solution. It is also believed that this is the first disclosure of a nanoparticle smaller than 20 nm that can envelop and retain a small molecule drug or molecular probe and release it in vivo in response to an external stimulus, such as energy. While it is conceivable that a larger particle can be used in this way, the use of a nanoparticle, with its accompanying advantages in vivo over macroscale particles, is not known to have been considered or successfully tested. Further, creation of a water-soluble nanoparticle delivery vehicle that comprises a cargo protected by a hydrophobic barrier provides the ability to delivery water-sensitive substances to sites of interest within an animal without, or substantially without, degradation, activation, or otherwise diminution of a characteristic of interest possessed by the substance provides advantages over other delivery vehicles known in the art. The numerous additional advantages provided by the nanoparticle delivery vehicle of the invention will be recognized by those practicing the invention.

### EXAMPLES

The invention will be further described with respect to implementation of nanoparticles in non-invasive diagnosis of vesicoureteral reflux (VUR). Those of skill in the art will immediately recognize that the concepts disclosed are applicable to other diseases, disorders, systems, organs, and tissues.

These examples relate to a unique new method to diagnose and monitor VUR. VUR is a common disorder in children. VUR is the improper flow of urine from the bladder up to the kidneys, leading to kidney infection, scarring, renal failure, and need for dialysis and renal transplant. The gold standard for diagnosis and surveillance is the placement of a catheter through the urethra, into the bladder, and injection of a contrast/imaging agent to visualize the flow of fluid up to the kidneys. Although such catheterization is relatively easily achieved in adults, the process is extremely traumatic to children and to their parents. Thus, physicians have altered clinical practice to minimize the number of tests obtained, and the NIH has established this problem as a national healthcare research priority (Geanacopoulos, M. Ed. Strategic Plan for Pediatric Urology. NIDDK-Research Progress Report. Washington: Government Printing Office, 2006, pp 25-29).

The application of nanotechnology to the development of new contrast agents for currently used imaging modalities will help create a novel system of detection that eliminates the traumatic catheterizations and minimizes radiation exposure. These Examples describe procedures for the preparation of a nanoparticle delivery vehicle in which an MRI contrast agent is anchored to the central core (“inner core region”) and the core particle is enveloped with a selectively removable periphery shell that masks the particle's MRI contrast properties until the shell is removed. Specifically, the Examples show: 1. flexible synthetic procedures to prepare multi-layer nanoparticles for bladder delivery of a masked MRI contrast agent that can be unmasked once it is delivered; and 2. optimization of the MRI contrast properties of the particles, and discussion of testing of the particles in vivo.

One objective of the investigations that led to this invention was to develop an MRI contrast agent for catheter-free detection of VUR, the retrograde flow of urine from the bladder into the kidneys, which appears in 50,000 new U.S. cases annually. VUR leads to hypertension, renal scarring, and renal failure, resulting in dialysis and renal transplantation. The Examples describe a study on the synthesis of a nanoparticle delivery vehicle that can be injected in aqueous solution and can carry a masked MRI contrast agent through the body to the bladder where it can be selectively unmasked. The details of this study enables design and synthesis of a diagnostic tool that will eliminate the need for catheterization and radiation exposure during VCUG.

VUR is a commonly occurring disease affecting greater than 10% of the general population (Atala, A; Keating, M. A. Vesicoureteral Reflux and Megaureter. In Campbell's Urology. P. C. Walsh, A . B. Retik, E. D. Vaughan, Jr., A. J. Wein, Eds. Sanders: Philadelphia, 2002; Bailey, R. Vesicoureteral reflux and reflux nephropathy. In: Diseases of the Kidney. Edited by R. Schrier and C. Gottschalk, pp. 747-783, 1988). In children with urinary tract infection the incidence is as high as 29% to 50% (Walker, R.; Duckett, J.; Bartone, F.; McLinn, P.; Richard, G. Screening School Children for Urologic Disease. Birth Defects 1977, 13, 399-407). When reflux coexists with urinary tract infection and intrarenal reflux, the child is at significant risk of renal scarring, which can lead to end-stage renal disease. The current management of VUR has evolved in response to several observations that have changed over the past 40 years (Walker, R. Vesicoureteral reflux. In: Adult and Pediatric Urology, Second Edition. J. Gillenwater, J. Grayhack, S. Howards et al., Eds. Chicago: Year Book Medical Publishers, vol. 2, pp. 1889-1920, 1992). 1. Approximately 80% of low-grade reflux cases resolves with medical management; higher grades resolve less frequently (Schwab, C. W.; Wu, H. Y.; Selman, H.; Smith, G. H. H.; Snyder, H. M.; Canning, D. A. Spontaneous Resolution of Vesicoureteral Reflux: a 15-Year Perspective. J. Urology 2002, 168, 2594-2599). 2. Sterile reflux does not cause renal damage but persistent reflux of infected urine can (Ransley, P.; Risdon, R. Renal Papillae and Intra-Renal Reflux in the Pig. Lancet 1974, 2, 1114; Ransley, P.; Risdon, R. Reflux Nephropathy: Effects of Antimicrobial Therapy on the Evolution of the Early Pyelonephritic Scar. Kidney International 1981, 20, 733-742; Ransley, P.; Risdon, R.; Godley, M. High Pressure Sterile Vesicoureteral Reflux and Renal Scarring: A Experimental Study in the Pig and Minipig. Contrib. Nephrol. 1984, 39, 320-343). While VUR can be managed with both medical and surgical treatment, the relative merits of each remain unclear. 3. Ureteroneocystostomy, the most common surgical strategy, is safe and effective, but it is reserved for patients in whom medical management is unsuccessful after a year. With these clinical tools, the disease can be safely and easily managed in most cases until it resolves. In any case, reliable and consistent diagnosis and surveillance of the disease is essential to its effective management. Treatment of VUR ensues with antibiotic prophylaxis with careful surveillance. A more aggressive surgical approach is used if this strategy is not effective. (Chang, Andy Y, Kirk, Jennifer, and Canning, Douglas A. “Endoscopic Approaches to the Treatment of Vesicoureteral Reflux.” Smith's Textbook of Endourology. Ed. Arthur D. Smith et al., BC Decker, Inc: Hamilton, Ontario, 2006. 795-806.)

Although sterile reflux is not highly problematic, persistent reflux of infected urine (e.g., in the case of UTI) can cause permanent renal damage. While VUR can usually be managed easily until it resolves, reliable surveillance of the disease is essential to its effective management. Diagnosis of VUR requires VCUG, a process in which an iodinated contrast agent is placed in the bladder through a catheter. This necessitates both urethral catheterization and X-ray irradiation of the gonads. It is an object of this invention to provide a catheter-free VCUG based on MRI that eliminates both the catheterization and gonadal radiation associated with VCUG. To do so, a selectively activated MRI contrast agent is provided, which is an agent produced by encapsulating a gadolinium-based MRI contrast agent in a nanoparticle, whereby its MRI contrast properties are masked until revealed by release of the agent from the capsule. The invention includes the necessary synthetic chemistry and nanotechnology needed for such an MRI contrast agent. As such, the invention provides for a VCUG procedure that is redesigned as an MRI-based exam wherein a masked MRI contrast agent is delivered to the bladder by normal excretion, then activated. The general approach to making and using a nanoparticle delivery vehicle according to the invention is sketched in FIG. 1. As can be seen, the invention includes a nanoparticle featuring a contrast agent in its core. The core is surrounded by an applied protective coating or shell to attenuate the MRI contrast of the gadolinium until the protective shell is removed in the bladder.

Use of an in vivo “revealable” detection agent is advantageous for many reasons. In VCUG, the flow of the contrast agent backwards from the bladder towards the kidneys must be detected. Because normal flow is from the kidneys to the bladder, prior activation would render the systemic delivery inappropriate, as the agent would be detectable when flowing normally from the kidneys to the bladder.

### Example 1

### Proof of Concept for the Realization of an Ultrasound-Activated MRI Contrast

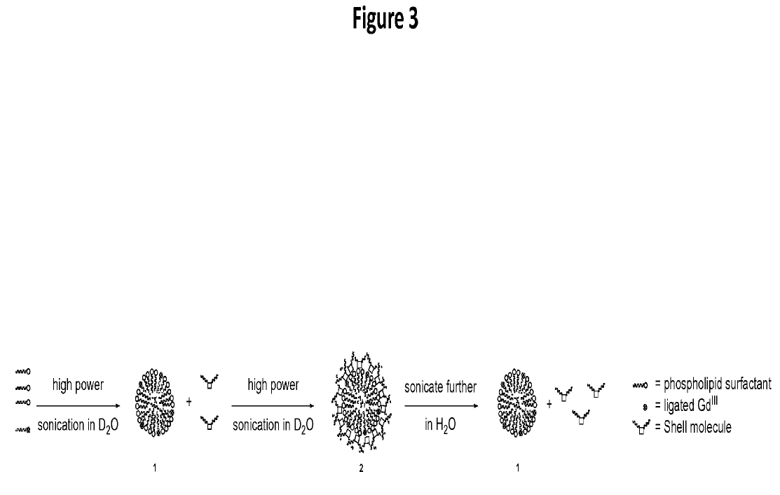

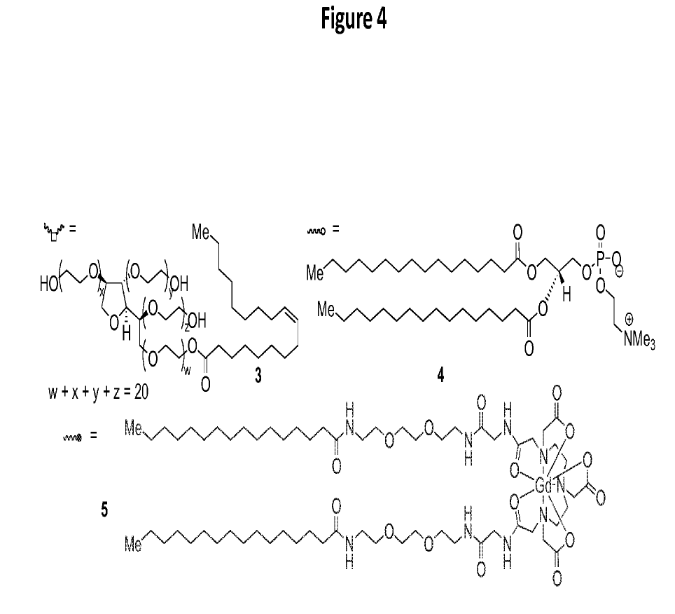

A non-covalently linked particle bearing a protective shell affords moderate MRI contrast upon activation by incubation with urea and activation with clinical ultrasound, but the nature of the core-shell linkage is critical to effecting contrast successfully. Our first trial revealed merits and opportunities for improvement in this plan. We prepared nanoparticles according to FIG. 3 based on commercial materials (FIG. 4) and a known procedure (Parac-Vogt, T. N.; Kimpe, K.; Laurent, S.; Piérart, S.; Vander Elst, L.; Muller, R. N.; Binnemans, K. Gadolinium DTPA-Monoamide Complexes Incorporated into Mixed Micelles as Possible MRI Contrast Agents. Eur. J. Inorg. Chem. 2004, 3538-3543). This first-pass experiment provided particles of 14 nm diameter with narrow dispersion. These particles were diluted in water in microcentrifuge vials and visualized by MRI using a Bruker Pharmacsan 7 T small animal scanner using a spin-echo (SE) experiment (repetition time/echo time, 345/11.5 msec). The results of this experiment showed modest contrast enhancement (about 4%) upon ultrasound activation.

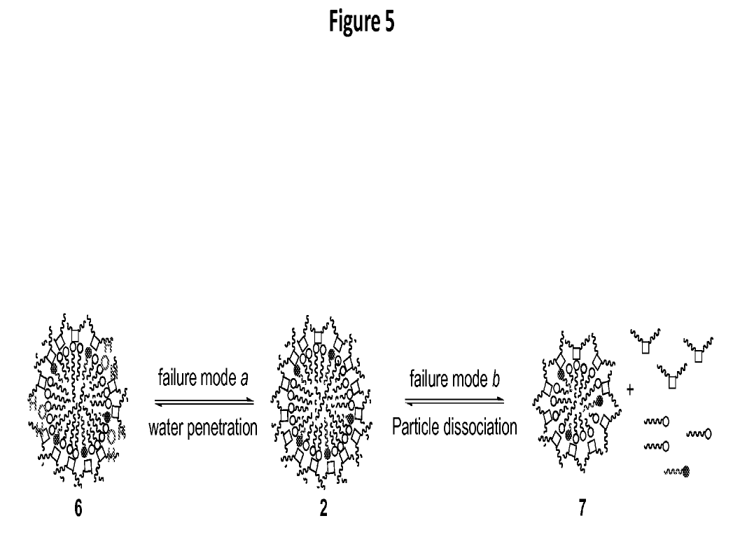

Although 4% contrast enhancement is encouraging, it is far from the most effective selectively activated MRI contrast agents reported to date. The two most likely reasons for low contrast enhancement for this experiment are sketched in FIG. 5. First (mode a), it appears that the shell, Tween-80, is not highly efficient at blocking water, thus the metal centers in the “unactivated” particles are well hydrated. It is also possible that the particles are dynamic (mode b), and that gadolinium species are partitioning in and out of the particle under the experimental conditions.

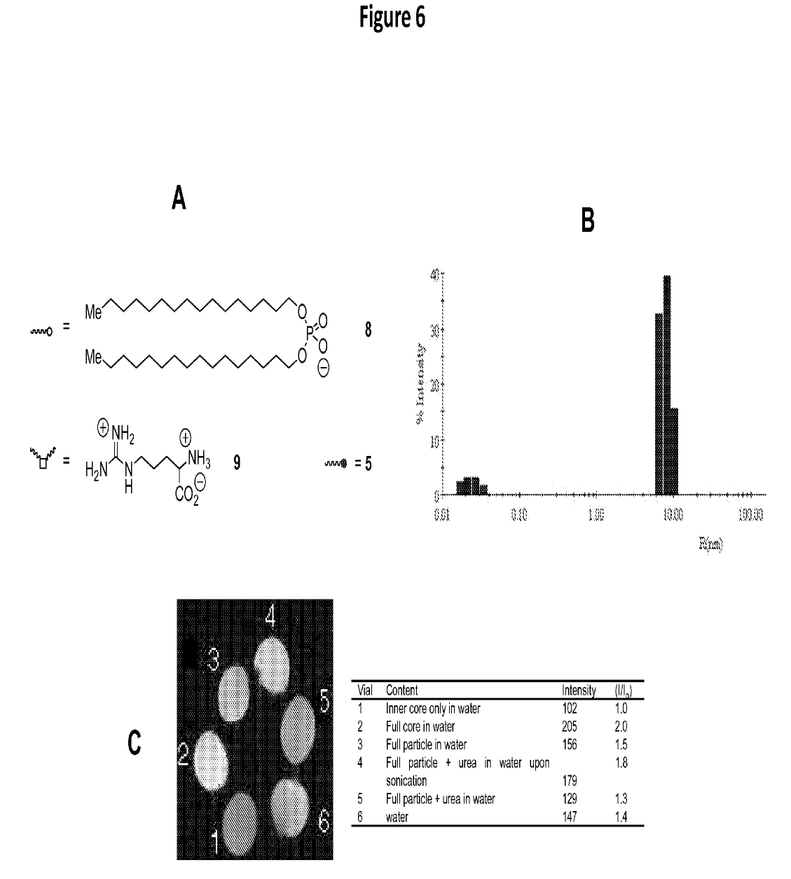

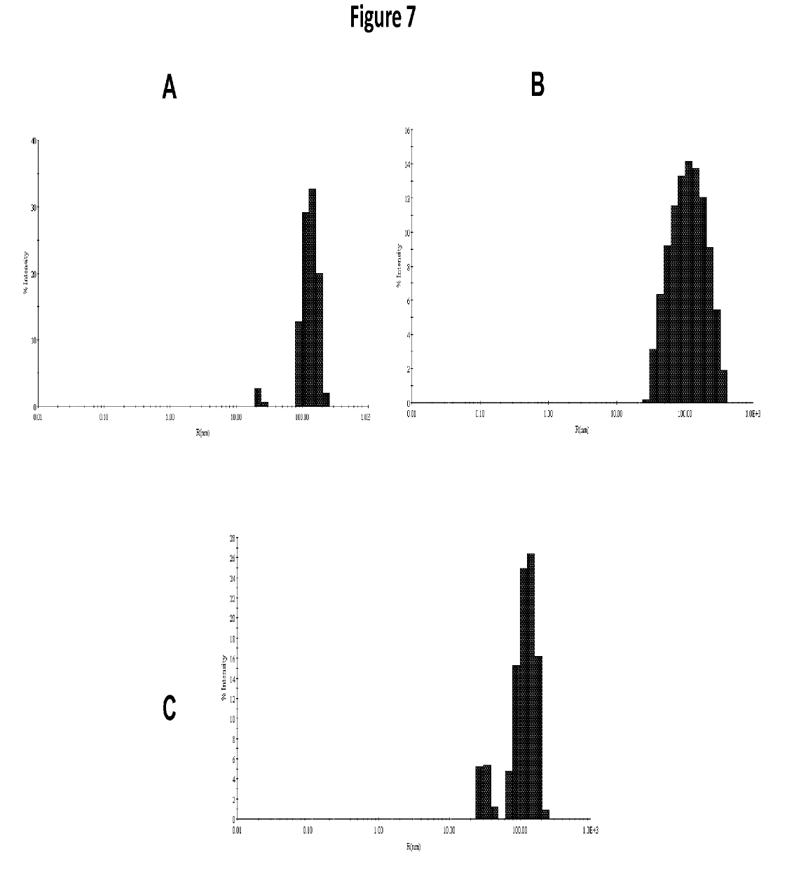

In a trial, we were able to obtain reasonable MRI contrast particle activation (FIG. 6). This was realized by incorporating a new design for the core-shell interaction wherein we rely on a guanidinium-phosphate doubly hydrogen-bound salt bridge to affix the shell to the particle core and where the phosphonic acid outer core region is covalently bound to a gold inner core. The design rationale of this selection is discussed in detail below.

A preliminary demonstration of our system is shown in FIG. 6. This figure shows an MRI image in which vials loaded with materials at various points in the particle synthesis are shown in a single image. The contrast differences between (a) positions 2 and 3 and (b) positions 3 and 4 respectively reveal that (a) attachment of the shell monomer attenuates the MRI contrast of the particle and (b) removal of the shell with ultrasound in an aqueous urea solution restores some of the original particle's contrast behavior. The DLS histogram illustrates that the diameter of these particles is centered around 10 nm.

### Example 2

### Non-Covalent Nanoparticles have a Size and Size Distribution that Depend on the Aqueous Environment

The shell governs size distribution in non-covalently linked particles. FIG. 7 shows the DLS data for micelles formed with gadolinium complex and dihexadecyl phosphate at 1.5 times the concentration used in FIG. 6. The data demonstrate that increasing the concentration increases the particle size. Also, FIG. 7 shows that size distribution broadens with addition of arginine and returns to a very similar distribution in the presence of urea. Beyond using different concentrations of dihexadecyl phosphate (compound 8 in FIG. 6) and gadolinium complex (compound 5 in FIG. 4), we tried other phosphonate and gadolinium complex surfactants to control the size and size distribution of the particles. The data we obtained show that size distribution broadens with addition of shell to core and returns to a very similar distribution in the presence of urea.

This Example indicates that non-covalent particles have a dynamic size and size distribution. The application and removal of a guanidinium-functionalized shell to our nanoparticles alters the mean diameter of the particles. This observation is consistent with a view that the surfactants in these particles are reorganizing under the conditions of shell application. This information was an important design consideration for further optimization of our system (below). The information lead us to design a nanoparticle with covalently attached Gd^{III} complex and surfactants to control size, size distribution, and particle stability.

### Example 3

### Proof of Principle for the Synthesis of Fluorinated Shell Molecules

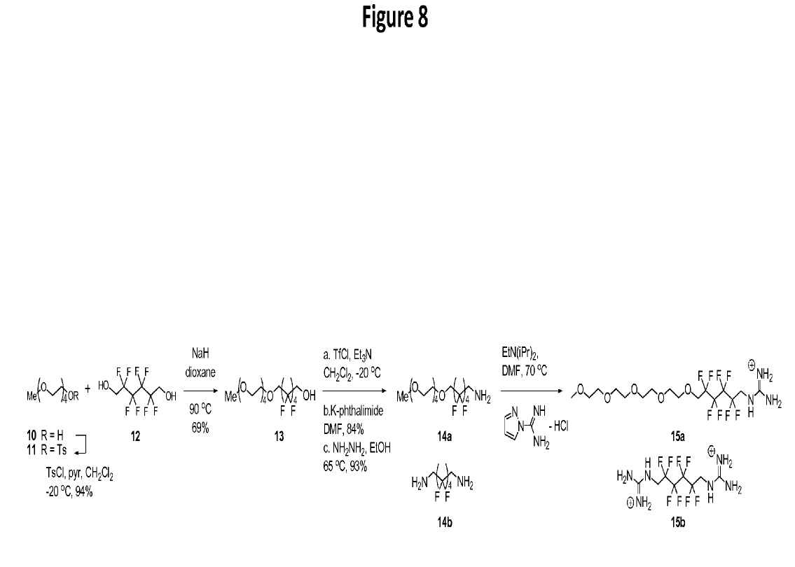

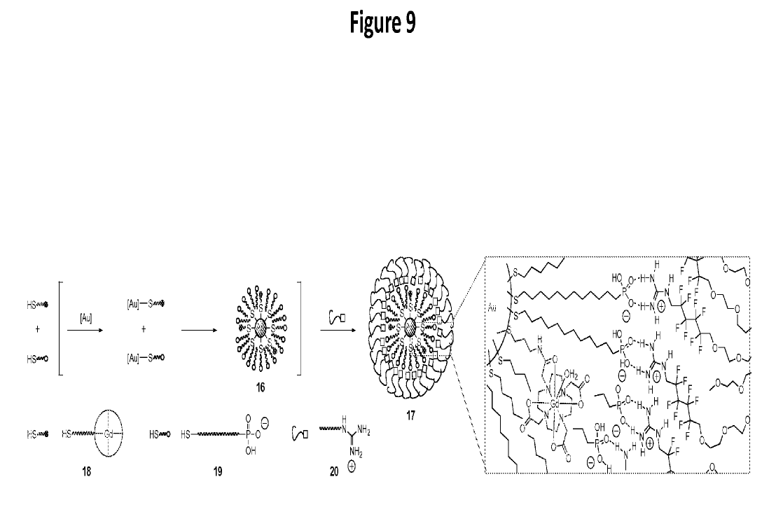

Using the data we collected above, we surmised that a shell molecule that features a water and lipid resistant fluorous region connected to a guanidinium head group would be advantageous. Generally, a fluorous spacer will 1. prevent this region of the shell from collapsing into the lipophilic interior of the particle, and 2. resist water partitioning through the shell, not unlike the commercial material available under the Gore-Tex® brand (W.L. Gore & Associates, Newark, Del.). Fluorous diols such as compound 12 in FIG. 8, are excellent building blocks for such molecules, and are available from Teflon® (E. I. Du Pont De Nemours and Company, Wilmington, Del.) by-products via an iodine-mediated oligomerization. The drawback of such starting materials is that functionalizing the alcohol group of such a fluorinated moiety as an electrophile is very difficult to achieve because of the electronegativity of the fluorine groups.

We have recently developed a synthesis for fluorous-functionalized shell molecules. FIG. 8 outlines our solution to the problem. The synthesis proceeds from commercially available tetraethyleneglycol monomethyl ether 10, which is converted to the tosylate and then reacted with fluorinated diol in the presence of sodium hydride to give the mono-alkylated product 13 in up to 69% yield, which is considerably higher than analogous procedures. Conversion of the alcohol to an amine functionality was then completed in two steps, via formation of the triflate, followed by displacement with potassium phthalimide, to form the carbon-nitrogen bond and reduction with hydrazine to form the amine 14a. This is the most streamlined synthesis of highly fluorinated amines that exists to the best of our knowledge. The final step in the synthesis of this shell molecule is the conversion of the amine to a guanidine end group, which was achieved via reaction with 1H-pyrazole-1-carboxamidine hydrochloride. The same technology was applied to the preparation of materials 14b and 15b, discussed below.

General Procedures: All air and water sensitive procedures were carried out either in a Vacuum Atmosphere glove box under nitrogen (2-10 ppm O₂ for all manipulations) or using standard Schlenk techniques under nitrogen when indicated. All reagents were purchased from Alfa Aesar or TCI and used without further purification. Dry solvents were obtained from EMD. All other solvents were reagent grade and used as received. Distilled water was purchased from Arrowhead, distilled and deoxygenated by purging with a stream of dry nitrogen.

Deuterated NMR solvents were purchased from Cambridge Isotopes Labs. Methanol-d₄ (CD₃OD) was used as received; chloroform-d (CDCl₃) was stored over 4 Å molecular sieved and potassium carbonate at room temperature. NMR spectra were obtained on a Varian Mercury 400 MHz (operating at 400 MHz for ¹H and at 100 MHz for ¹³C) spectrometer. All chemical shifts are reported in units of ppm and referenced to the residual ¹H solvent. ¹H NMR spectra were referenced to the residual protons in the deuterated solvent at 7.26 ppm for CDCl₃ and 4.48 ppm for CD₃OD. Data are reported as follows: chemical shift in the 6 scale; multiplicity (s: singlet, d: doublet, t: triplet, q: quartet, h: heptet, m: multiplet, br.: broad); integration; coupling constants (Hz); assignment. ¹³C NMR spectra were referenced to the solvent chemical shift at 77.0 ppm CDCl₃ and 49.15 ppm for CD₃OD. ¹⁹F NMR spectra were referenced to perfluorobenzene as an internal standard at −164.9 ppm relative to CFCl₃.

RP-LCMS was performed with a Shimadzu LC-MS using analytical column (Inertsil ODS-3; C18, 4.6×250 mm) with UV detection of product (λ=246 nm). The products were eluted utilizing isocratic 30% solvent A and 70% solvent B with flow rate of 0.6 mL/min where the solvent A is 0.1% TFA/H₂O and the solvent B is 0.1% TFA/CH₃CN. Mass spectra were obtained by electrospray ionization (ESI).

MALDI mass spectra were obtained on an Applied Biosystems Voyager spectrometer using the evaporated drop method on a coated 96 well plate. The matrix was anthracene. In a standard preparation, ˜1 mg analyte and ˜20 mg matrix were dissolved in a suitable solvent and spotted on the plate with a micro-pipetter. Standard C, H, N elemental analysis was performed by Desert Analytics Laboratory in Tucson, Ariz. or Galbraith Laboratories in Knoxville, Tenn. Fast atom bombardment (FAB) high resolution mass spectrum for compound 4 was collected by the University of California, Riverside Mass Spectrometry facility.

Experimental Details for New Compounds:

Triflate (2):

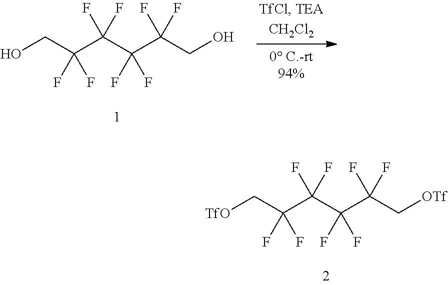

To a solution of diol 1 (5.0 g, 19.1 mmol) in dry dichloromethane (200 mL), trifluoromethanesulfonyl chloride (4.9 mL, 45.8 mmol) was added dropwise under a nitrogen atmosphere at room temperature. Triethylamine (10.7 mL, 76.3 mmol) was then added. A yellow precipitate was observed. The mixture was stirred at room temperature overnight. The solvent was then removed and the crude compound was dissolved in ethyl acetate (200 mL) and washed with water (100 mL×2). The phases were separated, and the organic phase was washed with 1M HCl (200 mL), NaHCO₃ (200 mL), and brine and then dried with MgSO₄. The solvent was removed under reduced pressure to yield yellow oil. It was then crystallized with 3:1 Hexanes:Ethyl Acetate to yield a white solid (9.4 g). Yield: 94%.

¹H NMR (400 MHz, CDCl₃): δ=4.82 (t, ³J_{H,F}=XX Hz, 4H, TfOCH₂CF₂).

¹⁹F NMR (376 MHz, CDCl₃): δ=126.1 (p, ³J_{F,F}=XX Hz, 4F, CF₂CF₂CH₂), −122.8 (apparent p, J˜XX Hz, 4F, CF₂CH₂O).

Phthalimide 3:

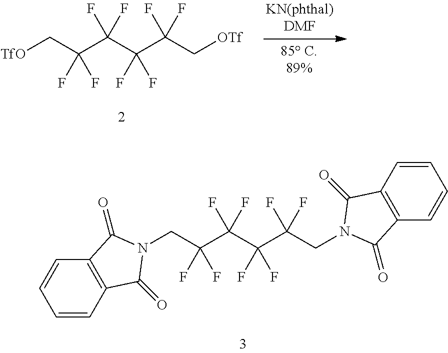

To a solution of 2 (3.0 g, 5.7 mmol) in dry N,N-dimethylformamide (300 mL), potassium phthalimide (2.53 g, 13.7 mmol) was added nitrogen atmosphere. The mixture was stirred at 85° C. overnight. The reaction was then cooled to room temperature and solvent was removed under reduced pressure. The crude compound was dissolve in ethyl acetate (200 ml) and undisolved white precipitate was removed by filtration. The solution was then washed with water (200 ml) and dried with MgSO₄. Solvent was removed to yield a white solid (2.6 g). Yield: 89%.

¹H NMR (400 MHz, CDCl₃): δ=7.93 (q, ³J_{H,H}=XX Hz, Ar, 4H), 7.78 (q, ³J_{H,H}=XX Hz, Ar, 4H), 4.38 (t, ³J_{H,F}=XX Hz, 4H, NCH₂CF₂).

¹³C NMR (100 MHz, CDCl₃): δ=

¹⁹F NMR (376 MHz, CDCl₃): δ=125.9 (p, ³J_{H,H}=XX Hz, 4F, CF₂CF₂CH₂), −118.7 (apparent p, J˜XX Hz, 4F, CF₂CH₂N).

Diamine 4:

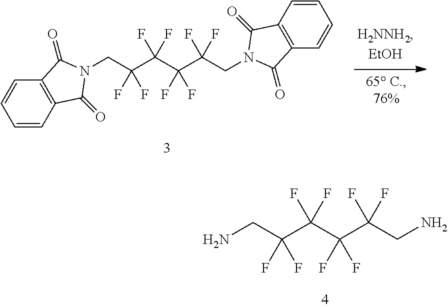

Compound 3 (2.3 g, 4.4 mmol) was treated with hydrazine (1.4 ml, 44.4) in 300 ml of pure ethanol under reflux overnight. After cooling to room temperature, the reaction mixture was filtered to remove the white precipitate. The solvent was then removed under vacuum. The crude product was then stirred in 200 ml of chloroform and re-filtered to remove any white precipitate. After the chloroform was removed, the product was obtained as a white solid (0.87 g) in 76% yield.

¹H NMR (400 MHz, CDCl₃): δ=3.26 (t, ³J_{H,H}=XX Hz, 4H, CH₂CF₂CF₂), 1.20-1.35 (b, 4H, NH₂CH₂CF₂).

¹³C NMR (100 MHz, CDCl₃): δ=

¹⁹F NMR (376 MHz, CDCl₃): δ=−127.0 (m, 4F, CF₂CF₂CH₂), −124.9 (m, 4F, CF₂CH₂NH₂) ppm.

PEG Tosylate (6):

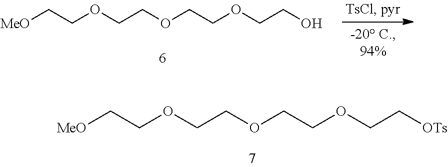

The same procedure from the literature was applied. To a solution of tetraethyleneglycol monomethyl ether (6) (10.0 g, 48.0 mmol) and pyridine (84 mL) in CH₂Cl₂ (170 mL), solid p-toluenesulfonyl chloride (22.0 g, 115.4 mmol) was added portion-wise at −20° C. under nitrogen and the resulting reaction mixture was stirred for 2 days at −20° C. Then, the reaction mixture was allowed to warm up to room temperature and water (200 mL) was added. The aqueous layer was extracted with CH₂Cl₂ (3×150 mL). The combined organic layers were dried over MgSO₄ and evaporated. The crude product was purified by flash chromatography (SiO₂; 1:1, EtOAc/Hexanes; R_{f}=0.3) to yield 7 as a colorless oil (16.4 g, 94%).

¹H NMR (400 MHz, CDCl3): δ=7.77 (d, ³J_{H,H}=XX Hz, 2H), 7.32 (d, ³J_{H,H}=XX Hz, 2H), 4.13 (t, ³J_{H,H}=XX Hz, 2H), 3.66 (t, ³J_{H,H}=XX Hz, 2H), 3.60 (s, 6H), 3.55 (s, 4H), 3.51 (t, ³J_{H,H}=XX Hz, 2H), 3.34 (s, 3H), 2.43 (s, 3H).

¹³C NMR (100 MHz, CDCl₃): δ=144.71, 132.94, 129.74, 127.89, 71.85, 70.65, 70.52, 70.50, 70.44, 70.43, 69.17, 68.59, 58.94, 21.56.

Fluorinated Alcohol 7:

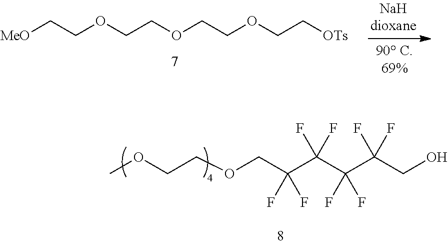

To a solution of diol 1 (21.7 g, 83.0 mmol) in dry dioxane (236 mL), NaH powder (1.7 g, 69.0 mmol) was added under nitrogen and stirred for 1 h at room temperature. A solution of 7 (10 g, 27.6 mmol) in dry dioxane (40 ml) was then added drop-wise. The mixture was heated at 90° C. and stirred overnight. Then the reaction was cooled down and quenched by water (100 mL) and then all the solvent was removed under reduced pressure. The crude compound was dissolved in diethyl ether (400 ml) and a white precipitate was removed via filtration. After solvent removal, the crude product was purified by flash chromatography (Ethyl acetate/Hexanes, 1:2 (R_{f}=0.5) to 5:1) to yield the monosubstituted product as a clear oil (8.6 g). Conversion: 100%. Yield: 69%.

¹H NMR (400 MHz, CDCl₃): δ=3.99 (m, 4H), 3.75 (m, 2H), 3.62 (m, 12H), 3.52 (m, 2H), 3.44 (t, 1H), 3.35 (s, 3H).

¹⁹F NMR (376 MHz, CDCl3): δ=127.2 (p, 2F), −127.1 (p, 2F), −125.7 (p, 2F), −123.0 (p, 2F) ppm.

16, 16, 17, 17, 18, 18, 19, 19-octafluoro-2,5,8,11,14-pentaoxaicosan-20-yl trifluoromethane sulfonate (8): To a solution of 7 (5.0 g, 11.1 mmol) in dry dichloromethane (30 mL), trifluoromethanesulfonyl chloride (3.5 mL, 33.1 mmol) was added dropwise under nitrogen. The mixture was then cooled to 0° C., and a solution of triethylamine (14.0 mL, 99.8 mmol) in dichloromethane (14 mL) was added dropwise, after which the solution turned yellow and a white precipitate was observed. The mixture was stirred at room temperature overnight. In order to obtain complete conversion, successive additions of trifluoromethanesulfonyl chloride (overall 4.7 mL, 44.2 mmol) and triethylamine (overall 18.6 mL, 133 mmol) were carried out over 2 days. The solvent was then removed under reduced pressure. The crude compound was dissolved in Ethyl acetate (200 mL) and the organic layer was washed with water (100 mL×2) and dried with MgSO4. The solvent was removed under reduced pressure to yield a yellow oil (6.5 g). Yield: 100%.

¹H NMR (400 MHz, CDCl₃): δ=4.80 (t, 2H), 4.03 (t, 2H), 3.77 (m, 2H), 3.64 (m, 12H), 3.53 (m, 2H), 3.36 (s, 3H).

¹⁹F NMR (376 MHz, CDCl₃): δ=−126.6 (h, 4F), −123.1 (p, 2F), −122.9 (p, 2F), −77.1 (s, 3F) ppm.

2-(16,16,17,17,18,18,19,19-octafluoro-2,5,8,11,14-pentaoxaicosan-20-yl)isoindoline-1,3-dione (9): Phthalimide potassium salt (4.12 g, 22.3 mmol) was added to a solution of 8 (6.5 g, 11.1 mmol) in 223 ml of DMF. The reaction was stirred at 65° C. overnight under nitrogen. The reaction was then cooled room temperature and solvent was removed under reduced pressure. 200 ml of CHCl₃ was added and a white precipitate was filtered. The solvent was removed under reduce pressure. The compound was then purified on a Silica gel column with 4:1 Ethyl acetate:Hexanes (Rf=0.4) to yield a clear oil (5.4 g). Yield: 84%.

¹H NMR (400 MHz, CDCl3): δ=7.92 (q, 2H), 7.78 (q, 2H), 4.35 (t, 2H), 4.03 (t, 2H), 3.77 (m, 2H), 3.64 (m, 12H), 3.53 (m, 2H), 3.36 (s, 3H).

¹⁹F NMR (376 MHz, CDCl3): δ=−126.8 (p, 2F), −126.5 (p, 2F), −123.1 (p, 2F), −119.3 (p, 2F) ppm.

16,16,17,17,18,18,19,19-octafluoro-2,5,8,11,14-pentaoxaicosan-20-amine (10): Compound 9 (3.8 g, 6.5 mmol) was treated with 2.05 mL (65.2 mmol) of hydrazine in 150 ml of pure ethanol at 65° C. and stirred overnight under nitrogen. The reaction was cooled to room temperature. White precipitate was filtered and washed with ethanol. The solvent was removed under reduced pressure. Then 200 ml of CHCl₃ was added and stirred for 30 minutes. More white precipitate was obtained and the precipitate was filtrated and the solvent was removed to yield the product as a light yellow oil (2.74 g) Yield: 93%.

¹H NMR (400 MHz, CDCl3): δ=4.00 (t, 2H), 3.76 (m, 2H), 3.63 (m, 12H), 3.53 (m, 2H), 3.36 (s, 3H) 3.23 (t, 2H). 13C NMR (100 MHz, CDCl3): δ=

¹⁹F NMR (376 MHz, CDCl3): δ=−127.2 (p, 2F), −126.9 (p, 2F), −124.8 (p, 2F), −123.2 (p, 2F) ppm.

1-(16,16,17,17,18,18,19,19-octafluoro-2,5,8,11,14-pentaoxaicosan-20-yl)guanidine (11):

It is believed that this is the first method developed to prepare guanidine-functionalized fluoroalkanes. The nearest example involved imidazole-functionalized fluoroalkanes. (Zeng, Z.; Phillips, B. S.; Xiao, J.-C.; Shreeve, J. M. Polyfluoroalkyl, Polyethylene Glycol, 1,4-Bismethylenebenzene, or 1,4-Bismethylene-2,3,5,6-Tetrafluorobenzene Bridged Functionalized Dicationic Ionic Liquids: Synthesis and Properties as High Temperature Lubricants. Chem. Mater. 2008, 20, 2719-2726.)

This Example 3 shows that we have surmounted the synthetic hurdles to preparing trifunctional molecules in which a fluorous core is decorated with a guanidinium head group and water-soluble tail.

### Example 4

### Development of Flexible Synthetic Procedures to Prepare Multi-Layer Nanoparticles that Meet Stated Criteria for Bladder Delivery and MRI Contrast Control

Because of our observation in Example 2, we approached this aim by assembling the particle's core around a rigid metallic center. In this strategy we covalently affix a gadolinium-containing contrast agent and surfactant molecules to mononuclear gold complexes, then allowed the gold to form a nanoparticle. We then applied the removable shell to this construct. Again, the working hypothesis was that the particles must be 1. easily prepared, 2. water-soluble, 3. minimally toxic, 4. gadolinium-containing, 5. of diameter below around 10 nm, 6. able to attenuate water partitioning to the and Gd^{III}, activated to allow water partitioning to the Gd^{III}. This Example explains the design of particles that meet these criteria, and explains the synthetic procedures that apply to their preparation.

A working imaging system is available with which optimization and validation studies can be performed. Specifically, we have achieved a greater than 25% contrast enhancement with our first-generation gold-centered nanoparticle delivery vehicle. When we apply our quantification system to published images of top “smart” MRI contrast agents, we observe off/on enhancement on the order of 100% (2-fold).

Examples 1 and 3 provide two key points justifying the feasibility of our approach: 1. We can realize reasonable contrast upon activation with a nanoparticle delivery vehicle; and 2. We have reasonable procedures to prepare more elaborate shell molecules. Example 2 offers important justification for our decision to switch to a covalently anchored particle scaffold.

Our original concept for an ultrasound-activated MRI contrast agent stemmed from a drug delivery strategy in which a delivery cargo was encapsulated in a vesicle surrounded by a phospholipid bilayer membrane. Such an approach has been used in various drug delivery applications. Moreover, detailed studies have been done on the efficacy of ultrasound-activated drug release from around 100 nm liposomes and the role of lipid packing in ultrasound-mediated drug release. The intrinsic nature of this construction, however, is that the width of a single phospholipid bilayer is around 6 nm. Thus, the smallest theoretical vesicle that could possibly form is of 12 nm diameter before any content is added. However, we recognized that, preferably for use in kidney studies, the particles should have a less than 10 nm diameter. We have now recognized that a single micelle strategy overcomes the size restriction issue and the gold core strategy allows excellent size control. More importantly, this strategy features covalent anchoring of all of the gadolinium containing species to the particle's center. That can avoid dissociation of the particle's constituent parts. The strategy has further appeal for its operational simplicity: this approach involves only a single step (one purification) from thiol compounds and gold colloids. Furthermore, gold nanoparticles are known to be non-toxic in vivo as drug delivery vehicles.

The particles described above are then surrounded by a water-soluble yet water impermeable shell. The shell is designed so that it can be removed selectively when desired. The design parameters of the shell compound are diagrammed in FIG. 10, Panel A. As can be seen, the shell molecule has a guanidinium head group to participate in hydrogen bonding with the phosphonates on the periphery of the core structure of the nanoparticle. This is attached to a hydrophobic “raincoat” region that will attenuate water partitioning into the interior of the particle so that H₂O exchange on gadolinium is suppressed. Incorporation of a fluorous phase (water and lipid immiscible) region will prevent both water partitioning and intercalation of the shell in to the interior of the particle. The hydrophilic ethylene oxide chain ensures solubility of the particle as well as the free shell molecule after liberation. Importantly, the poly(ethylene oxide) chain is known to be non-toxic and is known to generate non-toxic materials when conjugated to toxic components.

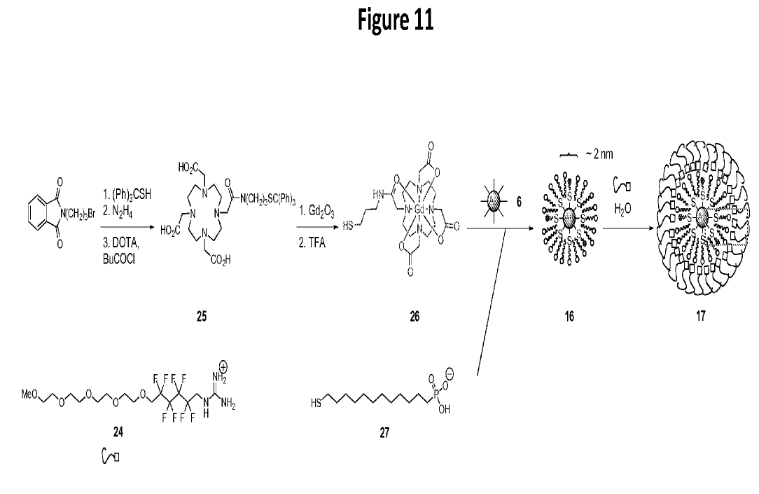

As outlined in FIG. 11, to make a suitable core structure, a mixture of thiol-terminated Gd^{III} and phosphonic acid surfactants in a ratio of 1:20 can be treated with gold colloids. Both of the required thiol monomers are known compounds. Compound 2 is commercially available, while compound 1 has been synthesized in the literature (Raghunand, N.; Jagadish, B.; Trouard, T. P.; Galons, J.-P.; Gillies, R. J.; Mash, E. A. Redox-Sensitive Contrast Agents for MRI Based on Reversible Binding of Thiols to Serum Albumin. Magn. Reson. Chem. 2006, 55, 1272-1280), and we have replicated this work in our lab. The resulting gold complexes can be reduced in situ to give gold nanoparticles. This facile procedure results in a well-defined, robust gold core, to which Gd^{III} and phosphonic acid moieties are covalently attached.

In the second step, the selectively removable shell must be attached to the periphery of the core structure of the nanoparticle. The shell molecules are attached to the phosphonic acid periphery by a guanidinium-phosphonate double hydrogen bonding system similar to the one viruses such as HIV-tat utilize to endocytose or otherwise enter cells. In this case, hydrogen bonding between the phosphonic acid periphery and a guanidine functionality at the terminus of the shell molecule will ensure tight binding, as has been observed in other micelle applications. In this application, the association constant is large, Kₐ˜10²-10⁴ M⁻¹ ((a) Fokkens, M.; Schrader, T.; Klrner, F.-G. A Molecular Tweezer for Lysine and Arginine. J. Am. Chem. Soc. 2005, 127, 14415-14421. (b) Williams, G.; Hardly, M. L. The Ionisation of Guanidine and of Amino-Acids in Sulphuric Acid. J. Chem. Soc. 1953, 2560-2563). In this scheme, hydrogen bonding and a Columbic attraction between the phosphonic acid periphery and a guanidinium group on the shell molecule will ensure tight binding. FIG. 10B illustrates this interaction. This specific interaction is cleaved in the bladder with clinical ultrasound. The dissociation of shell is driven to completion by the high concentration of urea (10 to 15 mM) in the bladder. More specifically, urea caps the phosphonic acid groups on the particle and help insure that the shell molecules do not re-associate (resulting in structure 23 of FIG. 10).

Exclusion of H₂O from the completed water-soluble particle is the most difficult hurdle in our design (failure mode a in FIG. 5), but the observation of contrast activity in FIG. 6 is very encouraging and suggests that this system can be optimized to an even higher contrast.

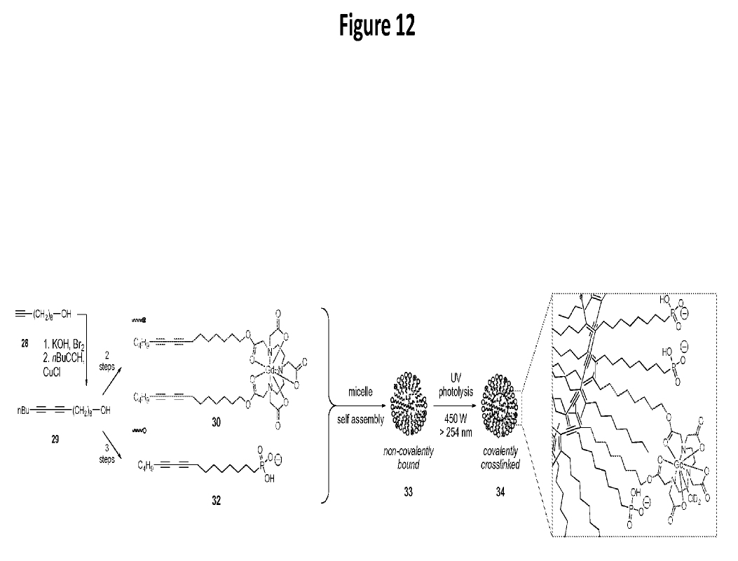

An alternative strategy for preparing a gold-based particle is to prepare a covalently bound particle core with a polymer cross-linking strategy as outlined in FIG. 12. In this design, UV photo-crosslinking of diyne moieties on the phosphonic acid and gadolinium surfactants forms a covalent network of bonds that prevents dissociation of surfactants from the core. Micelles self assemble from surfactants (again delivered in a 20:1 ratio) upon sonication. The resulting micelles can be irradiated with a 450 W mercury arc lamp to crosslink the diyne moieties covalently. These crosslinked structures are unable to dissociate or re-organize. The shell is then added to the crosslinked micelles using the same shell discussed above. The needed diyne phosphonic acid can be prepared by known methods, and ligated gadolinium compound 30 is available from materials in 4 steps, as follows.

Micelles are irradiated with a 450 W mercury arc lamp to covalently crosslink the diyne moieties (Guo, C.; Zhang, R.; Jiang, L.; Liu, T. Investigation of the Temperature Effect of a Mixed Vesicle Composed of Polydiacetylene and BODIPY 558. Colloids Surf., B 2007, 60, 41-45). The shell is then added to the cross-linked micelles, using the same shell molecule above. Synthesis of the needed diyne moieties is summarized in FIG. 12. Unsymmetrical diacetylenes 29 can be prepared by a modification of the Cadiot-Chodkiewicz coupling reaction of and alkynyl bromide (that can be obtained from bromination of commercially available10-Hydroxy-1-decyne (28) from TCI $17/g) with a terminal alkyne (Setzer, W. N.; Gu, X.; Wells, E. B.; Setzer, M. C.; Moriarity, D. M. Synthesis and Cytotoxic Activity of a Series of Diacetylenic Compounds Related to Falcarindol. Chem. Pharm. Bull. 2000, 48, 1776-1777). Then the phosphonic acid 32 can be prepared from 29 by first installing a toluenesulfonate ester, followed by reaction with diisopropyl hydrogen phosphate and hydrolysis to yield phosphonic acid 32 (Ostermayer, B.; Albrecht, O.; Vogt, W. Polymerizable Lipid Analogues of Diacetylenic Phosphonic Acids. Synthesis, Spreading Behaviour and Polymerization at the Gas-Water Interface. Chem. Phys. Lipid 1986, 41, 265-291). Ligated gadolinium compound 30 can be synthesized via esterificaion of the diacetylene alcohol 29 with a commercially available anhydride 9 and followed by refluxing with Gd₂O₃ in water (Tournier, H.; Hyacinthe, R.; Schneide, M. Gadolinium—Containing Mixed Micelle Formulations: A New Class of Blood Pool NRI/MRA Contrast Agents. Acad. Radiol., 2002, 9, S20-S28).

The photocrosslinking reaction outlined in FIG. 12 can be conducted as follows. A chloroform: methanol (4:1) solution containing gadolinium complex 30 and phosphonic acid 32 (1:20) in a ratio of 1 g surfactants to16 mL of solvent mixture can be concentrated to remove the organic solvents, and D₂O can be added into the flask. The flask can then be heated to 70° C. and probe sonicated for 20 minutes. The solution can then be put into a refrigerator and kept at 4° C. for 2 hours. With that, the micelle solution can be irradiated using a UV light (254 nm) for 30 minutes. Phosphonic acid 32 is similarly available.

### Example 5

### Testing and Optimization of the MRI Contrast Properties of the Particles Prepared Using the Synthetic Methods Developed in Example 4

We have disclosed and created a covalently linked nanoparticle that exhibits MRI contrast upon ultrasound activation, and we have developed convenient synthetic procedures for the preparation of similar particles. However, the particular starting materials and procedures described in Example 4 can be adjusted to provide different particles with different properties. The invention encompasses evaluation and testing of particles to provide alternative particles with alternative properties, as compared to the exemplary particles discussed above. Features that can be adjusted include, but are not limited to, size, solubility, metal content, shell properties, and synthesis steps. For example, adjustments in the total amounts and relative amounts of the gadolinium and phosphate starting materials that are used to fabricate the particle core can be made. Likewise, variations in the amounts and relative amounts of fluorous and PEG starting materials to alter water partitioning to the core and solubility can be made. The general utility of our system is proportional to the maximum level of contrast enhancement that we can realize, so optimization of the particles, for example to achieve an improvement in contrast of 2× or more, is envisioned through adjustment of various parameters. This Example discloses synthetic work to optimize the structure and composition of the exemplary nanoparticle delivery vehicle.

Gadolinium Loading. The ratio of Gd^{III} and phosphonic acid surfactants can be systematically varied in order to find the highest effective loading of Gd^{III} that allows to particle to function as designed. In Example 4, we chose to be begin with a 1:20 ratio because we want the surface to be predominantly covered with phosphonate groups to ensure dense coverage by the shell. However, that ratio might not be optimal under all conditions. It is a simple matter to vary the ratio and determine a suitable ratio for each of the various nanoparticles that can be made according to the present invention.

Tether Lengths and Topology. The lengths of the hydrocarbon “tethers” in the phosphonic acid- and gadolinium-functionalized thiols of the outer core can be systematically varied. The ratio of the length of the hydrocarbon spacers holding the caged gadolinium and the phosphonate moieties to the core can be a critical variable in contrast optimization. Further, contrast optimization may rely on the number of methylene groups in 26 and 27 (FIG. 11). It is a simple matter to vary these elements to find the optimal point for maximal contrast enhancement.

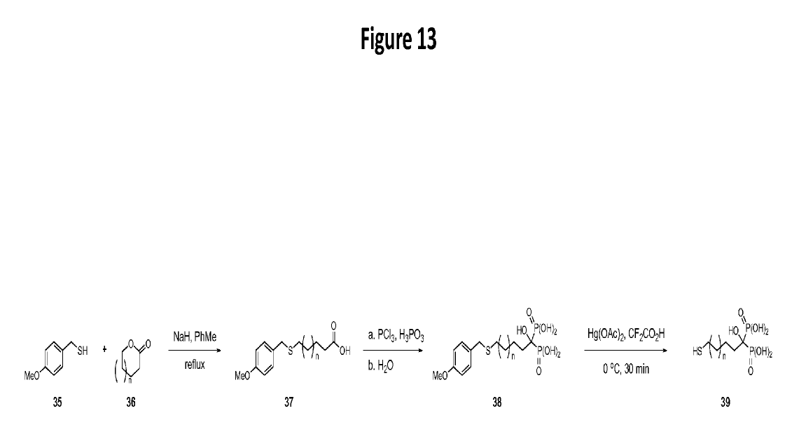

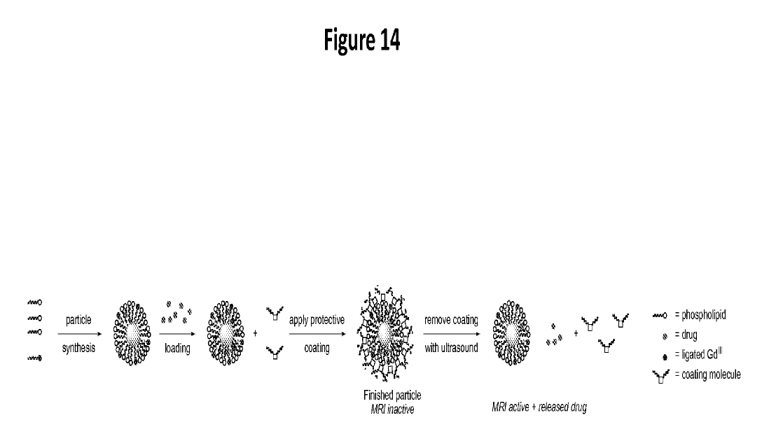

The invention encompasses a strategy for developing more dense periphery of phosphonic acid groups through the use of a branched phosphonic acid; a representative example is illustrated in FIG. 13. Thiol 35 is used to open any of a number of commercially available lactones according to a known procedure. The resulting compound is a carboxylate-terminated thioether, such as 37. Phosphorous acid is then used to convert this carboxylate to bisphosphonic acid 38. Cleavage of thioether 38 in the presence of mercury(II) and trifluoroacetic acid affords thiol-terminated surfactants 39. Importantly, 5-methoxybezylthioethers are known to be stable to HBr/HOAc, which indicates that this group will survive the corrosive conditions involved in the installation of bisphosphonate moiety (37 to 38).

Relative lengths of fluorous and PEG groups. It is difficult to predict, a priori, the minimum size the fluorous spacer that will be needed to provide an effective barrier to water partitioning. We have created nanoparticle delivery vehicles that comprise a C₄F₈ group as the hydrophobic region, but it is possibly that a larger fluorous region will be preferable and that a shorter fluorous region will provide acceptable results. Diols of the form HOCH₂(CF₂)ₙCH₂OH are available as byproducts from Teflon® production as mentioned above for n=4 (12), 5, and 6. The n=8 homolog is available from commercial diiodide I(CF₂)₈I through known methods. Systematic variation of fluorous diols can be performed to study of MRI contrast activity of particles prepared from these fluorous diols according to the various core structures that can be prepared according to the invention.

Similarly to the fluorous region, a systematic study of PEG units of different lengths and architectures can be performed. The key point here is to have enough PEG group to maintain water solubility of the particles, and to select shell molecules that avoid eliciting an immune response. Such parameters can easily be achieved.

Contrast enhancement in MRI imaging is not necessarily the most reliable (although most relevant) comparative tool for analysis of the effectiveness of MRI contrast agents. Thus one can also measure relaxivity of particles using an established procedure to quantify on/off selectivity in a way that is comparable both internally and with published data.

Effective size of the completed particles in solution can be directly measured by dynamic light scattering and TEM quickly and easily.

Testing for renal clearance is an important issue that can be determined in vivo or simulated in vitro. For in vitro simulation, Whatman Anotop filter membranes (20 nm filtration) can be used to control particle size. This serves two purposes. First, it can show that the data recorded by DLS accurately describes the effective solution diameter of the particles as they pass filtration in aqueous solution with their associated water of hydration. Further, by filtering out particles of large diameter, one can be assured that the MRI results observed are based on particles of clinically relevant sizes.

### Example 6

### Techniques to Analyze Nanoparticle Delivery Vehicles

In this Example, we describe testing procedures that can be used to evaluate the nanoparticles developed using the present invention. An in vitro test primarily involves procedures that we have demonstrated with data (DLS, MRI, etc.). An in vivo test describes an experiment performed in vivo to test some particles. In vitro particle evaluation and testing are listed here according to certain preferred design criteria.

Easily prepared. This criterion is built into the design of the surfactants, gadolinium sources, and shell molecules. Ease of synthesis is revealed instantly when the particles are made. Synthesis schemes that are inefficient or difficult to reproduce do not satisfy this preferred criteria, but may still be used to prepare nanoparticle delivery vehicles via a non-preferred way.

Water-soluble. Upon completion of a particle, its water solubility can be determined. Water insoluble particles are not preferred, as they will have in vivo properties that will be less desirable in most situations. The relevant concentration range of [Gd] that was used was 1-10 mM, based on literature protocol for MRI visualization Gd-containing nanoparticles. Nanoparticles that have insufficient solubility can be optimized for improved solubility.

Minimal toxicity. The toxicity of nanoparticles is an important consideration, particularly for nanoparticles designed for use in vivo. Initial toxicity evaluation will be based on known toxicity data for the various substances used. In vivo toxicity can be determined initially in vitro, then in an approved animal model.

Encapsulates Gd^{III}. This is built in to the synthetic plans. The presence of the metal can be quantified by elemental analysis or ICP-MS if needed.

Diameter below about 10 nm. At any time after preparing a nanoparticle delivery vehicle, its size can be measured by DLS. This rapid screening technique gives instant access to the size and size distribution of particles while in aqueous solution. One can further observe the size of particles by TEM, if desired.

Able to prevent or attenuate water partitioning to the Gd^{III}. This can be measured by MRI as illustrated in FIG. 6. Nanoparticle delivery vehicles can be assayed by relaxometry and MRI. Relaxivity of solutions containing particles can be determined by using NMR facilities or a relaxometer. The procedure for MRI measurement follows. Plastic microcentrifuge vials containing water and vials containing gadolinium solution on the order of 200 mM, are prepared. These can be imaged with a 7 T scanner (Bruker Pharmascan) using a head coil. The pulse sequences will be spin-echo (SE) (repetition time/echo time, 450/16 msec), fast multiplanar spoiled gradient echo (FMPSPGR) (repetition time/echo time, 100/3.5 ms; flip angle, 60°), or turbo fast low-angle shot (FLASH) (repetition time/echo time/inversion time, 11.0/4.2/300 ms; flip angle, 15°), depending on empirical observations. Data can be quantified as before.

Can be activated to allow water partitioning to the Gd^{III}. The removal of the shell can be monitored in several ways. First, DLS shows the change in particle size. The best measure of shell removal, however, will be relaxivity and MRI experiment described above. This will enable one to quantify effectively how well our contrast off/on “switch” works for each particle produced. NMR can also be used to corroborate these macroscopic observables with molecular events.

### Example 7

### In Vivo Experimental Design and Methods

Ability to unmask Gd^{III} in the bladder. The uniqueness and potential utility of the exemplary system disclosed in these Examples is its ability selectively to reveal Gd^{III} at the opportune moment and location. Parameters to consider and adjust for each particle and tissue/organ to be treated include: 1) attenuation of ultrasound energy as it penetrates through tissues to the compound; and 2) unforeseen interaction of the compound with animal cells and bodily fluids (e.g., urine and its components).

Juvenile (1.5 to 3 month old), female Sprague-Dawley rats can be utilized. Two rats can be anesthetized with isofluorane (1.5 to 3% with 2 liters/minute oxygen) and the bladders accessed through the urethras with 20-gauge angiocatheters. Based on in vitro experiments, varying dilutions of particles can be instilled into the bladders. The rats can then be scanned in a 7 T MRI before and after exposure to clinical ultrasound applied to the lower abdomen, immediately above the bladder. T₁ measurements can be performed based on the findings of the in vitro experiments delineated above. The duration of ultrasound exposure may be varied.

VUR Detection. The designed application of this nanoparticle tool is to detect vesicoureteral reflux. Thus, it is important to create VUR reliably and detect VUR with unmasked The following protocol can be used to validate the applicability of a particle of known contrast enhancement to visualization of VUR.

Vesicoureteral reflux can be induced in Sprague-Dawley rats when 1 mL of fluid is injected into the bladder. One can reproduce the creation of VUR in two anesthetized female, juvenile rats by catheterizing and injecting 1 mL of commercially available iodinated contrast (Cysto-Conray, Mallinckrodt Medical, Inc, St. Louis, Mo.) into the bladders via the 20-gauge angiocatheters. Fluoroscopy localizes the contrast at varying time points and confirms retrograde flow of urine/contrast into the ureters and kidneys. This process is easily and quickly performed and establishes the ability to induce VUR and the time points at which VUR occurs in this animal model. The experiment can be reproduced first using 1 mL of nanoparticles at optimal dilutions. After injection into the bladders, clinical ultrasound can be applied to the lower abdomens and bladders at varying durations, followed by MRI scanning Use of this test allows optimization of nanoparticles via an iterative process of testing, redesign, and retesting. Furthermore, after each of these nanoparticle experiments, the bladder contents can be collected and subjected to DLS analysis to determine unmasked versus masked particle ratios. The ratios quantify the effectiveness of the clinical ultrasound in unmasking the nanoparticle.

Intravenous injection. Catheterization needs to be avoided in order fully to obviate the trauma associated with the VCUG. While the above step examines the feasibility of unmasking Gd^{III} from the nanoparticle in vivo, it does not answer the questions of whether or not the particle can be eliminated through the kidney in an intact form and accumulate in the bladder in a sufficient amount to be unmasked and enhance MRI contrast.

Two female, juvenile rats can be injected with a compound useful according to the invention (5 mM of Gd^{III} in 150 mM NaCl hepes buffered solution at pH 7.4) and serial X-ray images taken with fluoroscopy at 5 minute intervals for 45 minutes to monitor the transit of the compound through the urinary tract as well as other organ systems. After which, clinical ultrasound can be applied to the lower abdomen and bladder and the animals imaged in a 7 T scanner Data available in the art shows that exposure to ultrasound at 45-minute time point after injection of the compound is suitable for detection of injected particles the kidney. Following the MRI scan, the bladders can be catheterized and urine collected. The nanoparticles in the urine can be analyzed and quantified to determine amount of elimination and percentage of nanoparticle fragmentation. This experiment gives a graphical read-out of the longevity of the shell. Regardless of where in the animal the contrast is activated, one will be able to see the enhanced contrast.

In vivo unmasking of the Gd^{III} plays a key role in functioning of the particles and methods of this embodiment of the invention. The energy required to remove the shell and expose Gd^{III} to water and urine might be attenuated by the anterior abdominal wall and bladder. This can be overcome with prolonged duration of clinical ultrasound exposure, or increased energy delivered with high frequency ultrasound or shockwaves. Another potential problem might be encountered if insufficient amount of urine is accumulated in the bladder. This can be addressed by: 1. Palpating for a full bladder prior to catheterizing the bladder and injecting the contrast agent, 2. Inducing diuresis by giving the rats water containing 5% glucose, which will cause greatly increased water consumption, and/or 3. Injecting the animals with furosemide, a diuretic, at a concentration of 0.5 mg/kg, although these methods will tend to decrease urea concentration.

Inducing vesicoureteral reflux reliably is necessary when the compound is injected intravenously. The presence of VUR is predicated on large volumes of fluid in the bladder. If unmasked Gd^{III} cannot be detected in the ureters and kidneys after activation with ultrasound, bladder volume can be increased with catheterization and injection of 0.5 to 1 mL of normal saline. Conversely, diuresis can be induced with flurosemide injection or feeding the rats 5% glucose water prior to intravenous compound injection.

Biodistribution of the injected nanoparticles is another concern. However, gold nanoparticles coated with ligated gadolinium moieties (˜2.4 nm diameter) are known to accumulate in the bladder within 45 minutes of intravenous injection. Although in the context of this embodiment of the invention, the gadolinium will be inactive until it reaches the bladder, gold nanoparticle biodistribution can be monitored via X-ray computed tomography due to their behavior as contrast agents for X-ray.

Simultaneous drug delivery and MRI contrast activation. The present invention further encompasses use of nanoparticles according to the invention to release highly toxic cancer drugs simultaneously with the activation of MRI contrast. This is a useful tool both for end-line clinical applications in which tumor tissue is visualized by MRI simultaneously with delivery of a therapeutic agent. It also has utility for studying therapeutic effects of anticancer agents for which biodistribution is unknown. Our conceptual strategy for this technology is sketched in FIG. 1. Following assembly of the core of the particle, the core is incubated with a lipophilic cytotoxin such as vinblastine, which enables partitioning of the agent into the hydrocarbon layer of the particle. Specifically regarding vinblastine, loading and release kinetics in DPPC (structure 4 of FIG. 4) based liposomes has been described. Finally, a fluorinated shell (e.g., structure 15a of FIG. 8) is attached. Because the fluorous region is both water- and lipid-immiscible, it should serve as an efficient barrier to simultaneously keep water out of the particle (because it is hydrophobic) and keep the lipophilic drug inside (because it is also lipophobic).

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only, with a true scope and spirit of the invention being indicated by the following claims.

1. A nanoparticle comprising:
a) a core structure connected through covalent bonds and having an organic material, a metal-containing material, or both, wherein the core structure comprises an inner core region for forming the core structure, and an outer core region for bonding the core structure to a shell structure;
b) a shell structure bound to the core structure, wherein the shell structure comprises, in sequential arrangement:
a binding region for binding to the core structure,
a hydrophobic region for protection of the binding region and core structure from hydrophilic substances, and
a hydrophilic region for rendering the nanoparticle soluble in hydrophilic environments; and
c) a cargo comprising a detectable agent, a bioactive agent, or a combination of the two, p1 wherein the nanoparticle has a diameter or a length in at least one dimension of 200 nanometers or less.

2. The nanoparticle of claim 1, wherein the nanoparticle has a diameter or a length in at least one dimension of 15 nanometers or less.

3. The nanoparticle of claim 1, wherein the inner core structure is connected to the outer core structure through covalent bonds.

4. The nanoparticle of claim 1, wherein the inner core region comprises gold atoms.

5. The nanoparticle of claim 1, wherein the cargo is an imaging agent comprising gadolinium.

6. The nanoparticle of claim 5, wherein the imaging agent is connected through covalent bonds to the core structure.

7. The nanoparticle of claim 6, wherein the imaging agent is connected through covalent bonds to a gold atom.

8. The nanoparticle of claim 1, wherein the cargo is non-covalently retained within the nanoparticle.

9. The nanoparticle of claim 1, wherein the cargo is an anti-cancer drug.

10. The nanoparticle of claim 1, wherein the shell further comprises a substance that targets the nanoparticle to a particular tissue or cell of an animal.

11. The nanoparticle of claim 10, wherein the substance is an antibody or a ligand for a cell-surface receptor.

12. The nanoparticle of claim 1, wherein the hydrophobic region of the shell structure comprises fluorine atoms.

13. The nanoparticle of claim 12, wherein the hydrophobic region of the shell structure comprises the following structure: (CF₂)ₙ, wherein n=2-10.

14. The nanoparticle of claim 1, which is spherical and has the following arrangement of elements from the interior to the exterior:
core structure inner core region;
core structure outer core region;
shell structure binding region;
shell structure hydrophobic region;
shell structure hydrophilic region; and
wherein the cargo is located in the core structure outer core.

15. A method of imaging animal tissue, said method comprising:
a) administering to a subject a nanoparticle comprising:
i) a core structure connected through covalent bonds and having an organic material, a metal-containing material, or both, wherein the core structure comprises an inner core region for forming the core structure, and an outer core region for bonding the core structure to a shell structure;
ii) a shell structure bound to the core structure, wherein the shell structure comprises, in sequential arrangement:
a binding region for binding to the core structure,
a hydrophobic region for protection of the binding region and core structure from hydrophilic substances, and
a hydrophilic region for rendering the nanoparticle soluble in hydrophilic environments; and
iii) an imaging agent,
wherein the nanoparticle has a diameter or a length in at least one dimension of 200 nanometers or less;
b) allowing adequate time for the nanoparticle to locate to a tissue of interest;
c) subjecting the nanoparticle to energy in an amount sufficient to break the bond between the core structure and the shell structure, causing the core structure and shell structure to dissociate; and
d) subjecting the tissue and imaging agent to an imaging technique.

16. The method of claim 15, wherein the energy is ultrasound and the ultrasound is applied at a level that is not harmful to animal tissue.

17. The method of claim 15, wherein the imaging technique is magnetic resonance imaging (MRI).

18.-26. (canceled)

27. A method of delivering a bioactive agent, a molecular probe, or both, to an animal tissue, said method comprising:
a) administering to an animal subject a nanoparticle comprising:
i) a core structure having an organic material, a metal-containing material, or both, wherein the core structure comprises an inner core region for forming the core structure, and an outer core region for bonding the core structure to a shell structure;
ii) a shell structure bound to the core structure, wherein the shell structure comprises, in sequential arrangement:
a binding region for binding to the core structure,
a hydrophobic region for protection of the binding region and core structure from hydrophilic substances, and
a hydrophilic region for rendering the nanoparticle soluble in hydrophilic environments; and
iii) a bioactive agent, a molecular probe, or both,
wherein the nanoparticle has a diameter or a length in at least one dimension of 200 nanometers or less;
b) allowing adequate time for the nanoparticle to locate to a tissue of interest; and
c) subjecting the nanoparticle to energy in an amount sufficient to break the bond between the core structure and the shell structure, causing the core structure and shell structure to dissociate.

28. (canceled)

29. The method of claim 27, wherein the energy is ultrasound and the ultrasound is applied at a level that is not harmful to animal tissue.

30. The method of claim 27, wherein the bioactive agent is a pharmaceutical.

31.-45. (canceled)
